(19) United States
(12) Patent Application Publication
(10) Pub. No.: US 2024/0277836 A1
(43) Pub. Date: Aug. 22, 2024

(54) Self-Amplifying RNA-Based VLP Vaccines

(71) Applicant: CHIMERON BIO CORPORATION, Philadelphia, PA (US)

(72) Inventors: Jolly Mazumdar Chendrimada, Philadelphia, PA (US); Thimmaiah Ponnappa Chendrimada, Philadelphia, PA (US)

(21) Appl. No.: 18/567,611

(22) PCT Filed: Jun. 9, 2022
(86) PCT No.: PCT/US2022/032876

§ 371 Date: Dec. 6, 2023

Related U.S. Application Data
(60) Provisional application No. 63/208,884, filed on Jun. 9, 2021.

(51) Int. Cl. A61K 39/215; A61K 39/00; A61P 37/04; C12N 7/00

(52) U.S. Cl. CPC A61K 39/215; A61P 37/04; C12N 7/00; A61K 2039/5258; A61K 2039/575; A61K 2039/6075; C12N 2740/11022; C12N 2740/11023; C12N 2770/20022; C12N 2770/20023; C12N 2770/20034; C12N 2770/36123

(57) ABSTRACT

The present disclosure provides compositions comprising an sa-RNA VLP vaccine (e.g. the VLP vaccine) that is capable of delivering a self-amplifying RNA to a target cell in a patient, and subsequently elicit an immune response in the patient, which immune response is sufficient to prevent or significantly decrease the duration of an infection by an infectious agent, such as SARS-CoV-2.

### CROSS-REFERENCE TO RELATED APPLICATIONS

This application claims the benefit of and priority to U.S. Provisional Application No. 63/208,884, filed Jun. 9, 2021, which is incorporated by reference in its entirety.

### SEQUENCE LISTING

The instant application contains a Sequence Listing with 17 sequences, which has been submitted via EFS-Web and is hereby incorporated herein by reference in its entirety. Said ASCII copy, created on Jun. 9, 2022, is named 46558_SEQListing_ST25.txt, and is 62 kilobytes (62 KB) in size.

### 1. Background

Globalization has an effect on both the transmission of pathogens and the availability of a pool of susceptible individuals. Global travel means that pathogens can travel around the world more easily. It also makes the pool of susceptible people larger. As a consequence, any new infectious disease, arising for example by mutation of an animal pathogen bacterial or viral (e.g. MERS, SARS, bird flu, Ebola, HIV etc.), has the opportunity to spread to susceptible individuals, and to be maintained within the human population for the long term.

An example of such a disease is the SARS-CoV-2, a recently emerged highly pathogenic human coronavirus. This disease has been declared a pandemic by the World Health Organization (WHO) and is having severe effects on both individual lives and economies around the world. Infection with SARS-CoV-2 is characterized by a broad spectrum of clinical syndromes, which range from asymptomatic disease or mild influenza-like symptoms to severe pneumonia and acute respiratory distress syndrome

In view of the high morbidity and mortality that can follow infection with bacterial and viral diseases, especially emerging pathogens, there is an urgent need for preventive vaccines.

### 2. Summary

Described herein are compositions relating to virus-like particles (VLPs) and methods for making and using the described VLPs. In certain embodiments, the compositions provide delivery of at least one immunogenic antigen of an infectious agent as a self-amplifying mRNA (sa-RNA) via a chimeric VLP (saRNA-VLP). In additional embodiments, the compositions include sa-RNA SARS-CoV 2 VLPs. In additional embodiments, the compositions include plasmids and cells used to produce the described VLPs.

Provided herein is a virus like particle (VLP), the VLP comprising:
a. an alphavirus replicon comprising a recombinant polynucleotide, wherein the polynucleotide encodes at least one polypeptide or fragment thereof which exhibits at least 80% identity to at least one immunogenic antigen of an infectious agent,
b. a retroviral gag protein,
c. a fusogenic envelope protein,
d. a retroviral packaging signal,
e. wherein the alphavirus replicon further comprises nonstructural proteins NSP1, NSP2, NSP3, and NSP4 and one or more subgenomic promoters, and
f. wherein the VLP does not contain an alphavirus structural protein.

Provided herein is a storage stable composition of virus like particles (VLPs), the VLPs comprising:
a. an alphavirus replicon comprising a recombinant polynucleotide, wherein the polynucleotide encodes at least one polypeptide or fragment thereof which is an immunogenic antigen of an infectious agent,
b. a retroviral gag protein,
c. a fusogenic envelope protein,
d. a retroviral packaging signal,
e. wherein the alphavirus replicon further encodes nonstructural proteins NSP1, NSP2, NSP3, and NSP4 and one or more subgenomic promoters, and
wherein the VLP does not contain an alphavirus structural protein;
wherein the composition is stable for at least about one-six months at 4° C.-10° C.

In certain embodiments, the infectious agent is a virus. In certain embodiments, the virus is a coronavirus. In certain embodiments, the coronavirus is SARS-CoV-2. In certain embodiments, the immunogenic antigen is a surface antigen. In certain embodiments, the surface antigen has at least 80% identity to a coronavirus spike protein, coronavirus membrane protein, coronavirus hemagglutinin esterase (HE), coronavirus envelope protein, or any combination thereof. In certain embodiments, the surface antigen has 80% identity to a coronavirus spike protein. In certain embodiments, the spike protein sequence has at least 90% identity with a coronavirus spike protein or ectodomain amino acids 1-1208 of SEQ ID NO: 1, or a combination thereof. In certain embodiments, the spike protein sequence comprises SEQ ID NO: 1 or the ectodomain region of amino acids 1-1208 of SEQ ID NO: 1 (which is equivalent to SEQ ID NO:17), or a combination thereof. In certain embodiments, the spike protein sequence is encoded by a codon optimized sequence of SEQ ID NO: 1; or a codon optimized ectodomain sequence of amino acids 990-1749 of SEQ ID NO: 1. In certain embodiments, at least one psi (Ψ) element is present upstream of the alphavirus replicon, and the psi element is from any member of the Retroviridae family. In certain embodiments, the at least one psi (Ψ) element is derived from Rous sarcoma virus (RSV).

In certain embodiments, the virus nonstructural proteins NSP1, NSP2, NSP3, and NSP4 are from Sindbis virus or Venezuelan equine encephalitis virus; and, optionally, the retroviral packaging signal is derived from Rous sarcoma virus (RSV). In certain embodiments, the alphavirus is a Venezuelan equine encephalitis virus. In certain embodiments, the VLP does not comprise or express a retroviral pol gene. In certain embodiments, the retroviral gag protein is derived from Rous sarcoma virus (RSV). In certain embodiments, the fusogenic envelope protein is a glycoprotein, or fragment or derivative thereof. In certain embodiments, the fusogenic envelope protein is VSV-G and is capable of binding to a target immune cell or antigen presenting cell. In certain embodiments, the target immune cell is a dendritic cell. In certain embodiments, the VLP is capable of binding to a target cell of a subject, after which the target cell is capable of expressing the immunogenic antigen of the infectious agent, which expression is capable of inducing an immune response, including a T cell response, against coronavirus in the subject. In certain embodiments, the immune response including a T cell response is induced by a dosing regimen comprising one or two administrations of the VLP, wherein the dose is sufficient to induce an immune response against coronavirus in a subject.

In certain embodiments, the dosing regimen comprises one administration of the VLP, wherein the one dose administration is sufficient to induce an immune response against the coronavirus in a subject.

Further provided herein is a pharmaceutical composition comprising the VLP of any of the previous embodiments. In certain embodiments, the composition further comprises a pharmaceutically acceptable carrier, diluent, adjuvant and/or additive, or any combination thereof. In certain embodiments, the composition is capable of inducing an immune response against coronavirus in a mammalian subject. In certain embodiments, following administration of the composition to the subject, the VLP is capable of inducing a T cell response against coronavirus in a mammalian subject. In certain embodiments, the coronavirus is SARS-CoV-2.

Further provided herein is a composition for use in diminishing or preventing at least one symptom of a coronavirus infection in a mammalian subject. In certain embodiments, said diminishing or preventing comprises inducing coronavirus-specific immunity against SARS-CoV-2 (COVID-19). In certain embodiments, the composition is for use in inducing cellular and or humoral immunity in a mammalian subject. In certain embodiments, the composition is for use in eliciting an immune response in a mammalian subject. In certain embodiments, said inducing or eliciting an immune response is an immune response against SARS-CoV-2 (COVID-19). In certain embodiments, the composition is for use in inducing neutralizing antibodies against SARS-CoV-2 in a mammalian subject.

Further provided herein is a method of diminishing or preventing a coronavirus infection in a mammalian subject comprising administering any of the VLP or pharmaceutical compositions described herein to the subject.

Further provided herein is a method of inducing cellular and or humoral immunity against a coronavirus in a mammalian subject, comprising administering any of the VLP or pharmaceutical compositions described herein to the mammalian subject.

Further provided herein is a method of eliciting an immune response against a coronavirus in a mammalian subject, comprising administering any of the VLP or pharmaceutical compositions described herein to the subject.

Further provided herein is a method of inducing neutralizing antibodies against SARS-CoV-2 in a mammalian subject, comprising administering any of the VLP or pharmaceutical compositions described herein to the subject. In certain embodiments, the method further includes inducing a T cell response against the coronavirus. In certain embodiments, the immune response is induced by a regimen comprising one or two administrations of the VLP or pharmaceutical composition. In certain embodiments, the immune response is induced by a regimen comprising one administration of the VLP or pharmaceutical composition. In certain embodiments, the coronavirus is SARS-CoV-2.

Further provided herein is a method of expressing a recombinant polynucleotide encoding an immunogenic antigen of an infectious agent in a subject, comprising administering any of the VLP or pharmaceutical compositions described herein to the subject. In certain embodiments, the VLP is capable of binding to a target cell, after which the target cell is capable of expressing the immunogenic antigen of the infectious agent, which expression is capable of inducing a cellular and/or humoral immune response to SARS-CoV-2 in the subject.

Further provided herein is a method of producing any of the VLPs described herein, comprising:
co-transforming a eukaryotic cell with:
   (a) a first plasmid comprising a polynucleotide sequence encoding the alphavirus replicon, wherein the polynucleotide encodes at least one polypeptide or fragment thereof which is an immunogenic antigen of an infectious agent;
   (b) a second plasmid comprising a polynucleotide sequence encoding a retroviral gag protein; and
   (c) a third plasmid comprising a polynucleotide sequence encoding the fusogenic envelope protein;
   (d) culturing the co-transformed eukaryotic cell under conditions suitable to cause each plasmid to produce its encoded product, thereby producing the VLP; and
   (e) isolating the VLP from the eukaryotic cell.

Further provided herein is a VLP produced by the method described above. Further provided herein is a method of diminishing or preventing a coronavirus infection in a mammalian subject comprising administering the VLP produced as described above to the subject. Further provided herein is a method of inducing cellular and or humoral immunity in a mammalian subject, comprising administering the VLP produced as described above, to the subject. Further provided herein is a method of eliciting an immune response in a subject, comprising administering the VLP produced as described above to the subject.

Further provided herein is a method of inducing neutralizing antibodies against SARS-CoV-2 in a subject, comprising administering the VLP produced as described above to the subject. In certain embodiments, the method further includes inducing a T cell response against the coronavirus. In certain embodiments, the T cell response is induced by a regimen comprising one or at least two administrations. In certain embodiments, the coronavirus is SARS-CoV-2 (COVID-19).

Further provided herein is a method of expressing a heterologous nucleic acid sequence in the host, comprising administering to a subject the VLP as described herein, wherein the VLP is capable of binding to a host cell, after which the host cell is capable of expressing the immunogenic antigen of the infectious agent, which antigenic expression on the host cell is capable of producing a cellular and/or humoral immune response to SARS-CoV-2 in the subject.

In certain embodiments, the VLP is stable at from the range of about 4° C.-10° C. for at least about one-six months. In certain embodiments, the VLP is stable for at least about six to nine months. In certain embodiments, the VLP is stable for at least about nine to twelve months. In certain embodiments, the VLP is stable at about −80° C. for at least about one year. In certain embodiments, the VLP is stable for about two years. In certain embodiments, the VLP is stable for about three years. In certain embodiments, the dose of the VLP is equivalent to about 100 pg to 1000 ng of RNA. In certain embodiments, the VLP is capable of binding to a host cell, after which the host cell is capable of expressing the immunogenic antigen of the infectious agent, which expression is capable of inducing a cellular and/or humoral immune response to Severe Acute Respiratory Syndrome (SARS-CoV), SARS-CoV-2, and variants of SARS-CoV-2 in the subject.

### 3. BRIEF DESCRIPTION OF THE DRAWINGS

These and other features, aspects, and advantages of the present invention become better understood with regard to the following description, and accompanying drawings, where:

FIG. 1: Delivery of the Viral Antigen as Self-Amplifying mRNA via a chimeric VLP. The viral-like protein (VLP) delivery particle expresses the VSV-G protein on its surface, that will target, in general, to immune cells. The VLP's payload is one or more viral antigens which will be delivered as mRNA.

FIG. 2: Design of the sa-RNA SARS-CoV-2 VLP vaccine. The VLP is typically generated by utilizing three separate plasmids encoding for the following: an envelope protein, e.g. VSV-G protein, a capsid protein, e.g. GAG protein, and encapsulating the RNA encoding either the full-length spike protein (CB-106) or the spike protein ectodomain region (CB-107).

FIG. 3A: Amino acid sequence of the Covid-19 spike glycoprotein (S-protein) (SEQ ID NO:1). Also shown in FIG. 3A-1 is the Codon optimized nucleotide sequence of the full length Covid-19 spike glycoprotein (S-protein) (SEQ ID NO:2). (Genbank ID NC_045512.2). SP—signaling protein; NTD—N-terminal domain; RBD—receptor binding domain; S2—S2 domain; TM—transmembrane region; and CT—Cytoplasmic tail.

FIG. 3B: Covid-19 spike domain regions: signal domain (SEQ ID NO:3), N-terminus domain (SEQ ID NO:4), and receptor binding domain (SEQ ID NO:5) (Genbank ID 43740568).

FIG. 3C: Amino acid sequence of the Covid-19 spike glycoprotein (S-protein) regions: S2 domain (SEQ ID NO:6), transmembrane region (SEQ ID NO:7) and cytoplasmic tail (SEQ ID NO:8).

FIG. 3D: Nucleotide sequence of the ectodomain COVID-19 spike glycoprotein (S-protein) ectodomain region of CB-107 (SEQ ID NO:9). FIG. 3E: Amino acid sequence elements from CB-107 (SEQ ID NO:10-13) (see FIG. 3G for full CB-107 amino acid sequence, SEQ ID NO:17).

FIG. 3F: polynucleotide sequences of the South African Variant RBD domain (SEQ ID NO:14), example of a suitable Psi element (SEQ ID NO:15), and an exemplary subgenomic promoter (SEQ ID NO:16).

FIG. 3G: Amino acid sequence of CB-107 (S-protein ectodomain) (SEQ ID NO:17) and lower panel schematic showing CB-106 and CB-107. SP—signaling protein; NTD—N-terminal domain; RBD—receptor binding domain; S2—S2 domain; TM—transmembrane region; and CT—Cytoplasmic tail.

FIG. 4: Confirmation of Infection of Immune Cells by VSV Particle In Vitro. THP1 human monocyte cells were transduced with a test VLP containing mCherry fluorescent reporter protein as the gene of interest. Fluorescence signal was assessed 48 hours after transduction.

FIG. 5: Study design. sa-RNA SARS-CoV-2 VLP vaccine candidates were tested in young naïve female Balb/c mice in a Prime and Boost design. The sa-RNA SARS-CoV-2 VLP vaccine was tested at multiple dose levels. Both vaccine candidates are active and were well tolerated. Effective neutralizing antibody production was observed in a prime and boost setting.

FIGS. 6A-6C are graphs showing the generation of neutralizing antibodies against SARS-CoV-2 by mice following vaccination with sa-RNA SARS-CoV-2 VLP candidates.

FIGS. 7A-7D are graphs illustrating the Th1/Th2 skew in response to SARS-CoV-2 VLP-saRNA vaccine. IgG (FIG. 7A), IgG1 (FIG. 7B) and IgG2a (FIG. 7C) responses in mice vaccinated with VLP-saRNA. N=5 animals. The mice demonstrated a Th1 bias (FIG. 7D) Data shown as Mean±SD. Significance compared to antigen control was determined by performing a one-way ANOVA; **p<0.001, ***p<0.0001.

FIG. 8 is a graph illustrating temperature stability of the sa-RNA SARS-CoV-2 VLP vaccine at 4° C. over the course of twelve weeks.

FIGS. 9A-9B illustrate safety tox studies in rabbits. A human equivalent efficacious dose of the sa-RNA SARS-CoV-2 VLP was tested in rabbits using a prime+boost design. There was no impact on body weight gain, food consumption (FIG. 9A) and other parameters measured (FIG. 9B).

### 4. DETAILED DESCRIPTION

### 4.1. VLPs with Self-Amplifying RNA for Eliciting an Immune Response Against or Preventing Viral-Mediated Infection and Related Conditions, Including Against Coronaviruses

While not wishing to be bound by theory, the present disclosure is based at least in part on the ability of an effective amount of an sa-RNA VLP vaccine (e.g. the VLP vaccine) to deliver self-amplifying RNA to a target cell in the patient, and subsequently elicit an immune response in the patient, which immune response is sufficient to prevent or significantly decrease the duration of an infection by an infectious agent, such as SARS-CoV-2, as described herein below.

### 4.2. Definitions

Unless defined otherwise, all technical and scientific terms used herein have the meaning commonly understood by one of ordinary skill in the art to which the invention pertains.

As used herein, the terms “patient” and “subject” are used interchangeably and may be taken to mean any living organism which may be treated with compounds of the present invention. As such, the terms “patient” and “subject” include, but are not limited to, any non-human mammal, primate and human.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. The terms “treat”, “treatment”, and the like regarding a state, disorder or condition may also include (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms.

As used herein “preventing” a disease refers to inhibiting the full development of a disease.

The term “biological sample” refers to any tissue, cell, fluid, or other material derived from an organism (e.g., human subject). In certain embodiments, the biological sample is serum or blood.

“Antibody” as used herein encompasses polyclonal and monoclonal antibodies and refers to immunoglobulin molecules of classes IgA (e.g., IgA1 or IgA2), IgD, IgE, IgG (e.g., IgG1, IgG2, IgG3 and IgG4) or IgM, or fragments, or derivatives thereof, including without limitation Fab, F(ab′)2, Fd, single chain antibodies, diabodies, bispecific antibodies, bifunctional antibodies, humanized antibodies, and various derivatives thereof.

As used herein, the term “neutralizing antibody” refers to an antibody that binds to a pathogen (e.g., a virus) and interferes with its ability to infect a cell. Non-limiting examples of neutralizing antibodies include antibodies that bind to a viral particle and inhibit successful transduction, e.g., one or more steps selected from binding, entry, trafficking to the nucleus, and transcription of the viral genome. Some neutralizing antibodies may block a virus at the post-entry step.

The term “immune response” refers to a response of a cell of the immune system (e.g., a B-cell, T-cell, macrophage or polymorphonucleocyte) to a stimulus such as an antigen (e.g., a viral antigen). Active immune responses can involve differentiation and proliferation of immunocompetent cells, which leads to synthesis of antibodies or the development of cell-mediated reactivity, or both. An active immune response can be mounted by the host after exposure to an antigen (e.g., by infection or by vaccination). Active immune response can be contrasted with passive immunity, which can be acquired through the transfer of substances such as, e.g., an antibody, transfer factor, thymic graft, and/or cytokines from an actively immunized host to a non-immune host.

As used herein in connection with a viral infection and vaccination, the terms “protective immune response” or “protective immunity” refer to an immune response that that confers some benefit to the subject in that it prevents or reduces the infection or prevents or reduces the development of a disease associated with the infection. As an example, the presence of SARS-CoV-2 neutralizing antibodies in a subject can indicate the presence of a protective immune response in the subject.

“Virus-like particle” (VLP), as used herein, refers to a structure resembling a virus particle. In preferred embodiments, a VLP embodies a structure containing alphavirus replicon encoding genes of interest packaged within a retroviral GAG capsid protein shell and with at least one full or partial fusogenic protein displayed on the surface of the particle. A virus-like particle as provided herein lacks all or part of the replicative components of the viral genome, i.e. does not contain any alphavirus structural proteins and does not contain a retroviral polymerase.

The terms sa-RNA VLP vaccine, “VLP vaccine composition”, or “VLP vaccine”, which are used herein interchangeably, refer to an sa-RNA VLP composition comprising a replicon encoding at least one immunogenic and/or antigenic component that is capable of inducing an immune response in a subject (e.g., humoral and/or cellular response). In certain embodiments, the immune response is a protective immune response. The VLP vaccine may be administered for the prevention or treatment of a disease, such as an infectious disease.

As used herein in connection with various recombinant VLPs, the term “chimeric” refers to VLPs comprising a mixture of non-structural components from one viral family (i.e. alphavirus non-structural proteins) and an additional component from a different viral family, e.g. a Psi element and GAG protein from any of the viruses belonging to the family Retroviridae.

The term “fusogen” or “fusogenic molecule” is used herein to refer to any molecule that can trigger membrane fusion when present on the surface of a virus particle. A fusogen can be, for example, a protein (e.g., a viral glycoprotein) or a fragment or derivative thereof.

The term “replication-competent” is used herein to refer to viruses (including wild-type and recombinant viral particles) that are capable of infecting and propagating within a susceptible cell.

The term “replication incompetent” is used herein to refer to VLPs that are not capable of propagating within a susceptible or target cell. Such a VLP can bind to, fuse and transfer its nucleic acid payload into the target cell, but has no viral replication components or structural proteins to assemble or be capable of viral replication. For example, replication incompetent VLPs do not comprise or express a retroviral pol gene.

The term “encoding” can refer to encoding from either the (+) or (−) sense strand of the polynucleotide for expression of a desired protein.

Antigen-providing mRNA: An antigen-providing mRNA as used herein may typically be an mRNA, having at least one open reading frame that can be translated by a cell or an organism provided with that mRNA. The product of this translation is a peptide or protein that may act as an antigen, preferably as an immunogen. The product may also be a fusion protein composed of more than one immunogen, e.g. a fusion protein that consist of two or more epitopes, peptides or proteins derived from the same or different virus-proteins, wherein the epitopes, peptides or proteins may be linked by linker sequences.

Antigen or antigenic polypeptide: In the context of the present invention “antigen” refers typically to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide (e.g. polypeptide) or protein, or fragment which is capable of being presented to T-cells. As provided herein, an antigen may be the product of translation of a provided nucleic acid molecule, preferably an mRNA as described herein. In this context, fragments, variants and derivatives of peptides and proteins comprising at least one epitope are understood to function as antigens.

Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed. The term percent “identity” or percent “homology,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. For purposes herein, percent identity and sequence similarity is performed using the BLAST algorithm, which is described in Altschul et al., J. Mol. Biol. 215:403-410 (1990). Software for performing BLAST analyses is publicly available through the National Center for Biotechnology Information (on the world wide web at: ncbi.nlm.nih.gov/).

In some cases, the identity between a reference sequence (query sequence, e.g., a sequence of the disclosure) and a subject sequence, also referred to as a global sequence alignment, can be determined using the FASTDB computer program. In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

The term “effective” applied to dose or amount refers to that quantity of a compound (e.g., a recombinant VLP) or composition (e.g., pharmaceutical, vaccine or immunogenic and/or antigenic composition) that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

As used herein, the phrase “a subject in need thereof” means a human or non-human animal that exhibits one or more symptoms or indicia of a disease or disorder associated with a coronavirus infection, and/or who is at risk of developing a disease or disorder associated with an infection. In certain embodiments, the coronavirus is SARS-CoV-2. In certain embodiments, the disease or disorder is COVID-19. In certain embodiments, the COVID-19 disease symptoms include, but are not limited to, fever, cough, shortness of breath, pneumonia, acute respiratory distress syndrome (ARDS), acute lung syndrome, loss of sense of smell, loss of sense of taste, sore throat, nasal discharge, gastro-intestinal symptoms (e.g., diarrhea), organ failure (e.g., kidney failure and renal dysfunction), septic shock and death in severe cases.

The terms “individual” or “subject” or “patient” or “animal” refers to humans, veterinary animals (e.g., cats, dogs, cows, horses, sheep, pigs, etc.) and experimental animal models of diseases (e.g., mice, rats, ferrets, monkeys, etc.). In a preferred embodiment, the subject is a human.

The terms “nucleic acid”, “polynucleotide” and “nucleotide” are used interchangeably and encompass both DNA and RNA, including positive- and negative-stranded, single- and double-stranded, unless specified otherwise.

The phrase “pharmaceutically acceptable”, as used in connection with compositions described herein, refers to molecular entities and other ingredients of such compositions that are physiologically tolerable and do not typically produce untoward reactions when administered to a subject (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “Coronavirus” as used herein refers to the subfamily Coronavirinae within the family Coronaviridae, within the order Nidovirales. Based on the phylogenetic relationships and genomic structures, this subfamily consists of four genera: Alphacoronavirus, Betacoronavirus, Gammacoronavirus and Deltacoronavirus. The alphacoronaviruses and betacoronaviruses infect only mammals. The gammacoronaviruses and deltacoronaviruses infect birds, but some of them can also infect mammals. Alphacoronaviruses and betacoronaviruses usually cause respiratory illness in humans and gastroenteritis in animals. The three highly pathogenic viruses, SARS-CoV-2, SARS-CoV and MERS-CoV, which cause severe respiratory syndrome in humans. The other four human coronaviruses, HCoV-NL63, HCoV-229E, HCoV-OC43 and HKU1, induce only mild upper respiratory diseases in immunocompetent hosts, although some of them can cause severe infections in infants, young children and elderly individuals. Additional non-limiting examples of commercially important coronaviruses include transmissible gastroenteritis coronavirus (TGEV), porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus. Reviewed in Cui et al., Nature Reviews Microbiology, 2019, 17:181-192; Fung et al., Annu. Rev. Microbiol., 2019, 73:529-557.

Severe acute respiratory syndrome beta coronavirus 2 and SARS-CoV-2 (COVID-19) are used interchangeably herein. In certain embodiments, the virus is SARS-CoV-2, also referred to as nCoV-2, nCoV2 or 2019-nCoV. The terms “nCoV2”, “nCoV-2”, “SARS-CoV-2” and “SARS-CoV-2 (COVID-19)” are used interchangeably herein. In particular embodiments, the sa-RNA VLP vaccine prevents severe acute respiratory syndrome (SARS) in the patient/subject.

### 4.3. Storage Stable sa-RNA VLPs With a Replicon Payload Encoding At Least One Immunogenic Antigen of an Infectious Agent

Aspects provided herein include a virus like particle (sa-RNA VLP), the sa-RNA VLP comprising:
a. an alphavirus replicon comprising a recombinant polynucleotide, wherein the polynucleotide encodes at least one polypeptide or fragment thereof which exhibits at least 80% identity to at least one immunogenic antigen of an infectious agent,
b. a retroviral gag protein,
c. a fusogenic envelope protein,
d. a retroviral packaging signal,
e. wherein the alphavirus replicon further comprises nonstructural proteins NSP1, NSP2, NSP3, and NSP4 and one or more subgenomic promoters, and

wherein the sa-RNA VLP does not contain an alphavirus structural protein.

In particular embodiments, the sa-RNA VLP vaccine provides the subject with an immune response (e.g. induces production of neutralizing antibodies in the patient/subject) that prevents or lessens coronavirus disease 2019 (COVID-19).

### Sa-RNA Virus-Like Particles

sa-RNAVLPs described herein will generally have the following features: they will comprise one or two RNA molecules encoding an alphavirus replicon which encodes at least one antigenic component of an infectious agent; they will have a viral core comprising a retroviral gag protein from any of the Retroviridae family, or, in some embodiments, a gag fusion protein; they will have a surface protein to facilitate fusion with a cell, and they will not contain a polynucleotide that encodes an alphavirus structural protein, and will be replication incompetent.

The VLPs described herein will be useful in transducing cells in order to express at least one antigenic component of an infectious agent. Accordingly, the described VLPs may incorporate one or two alphavirus-based RNA polynucleotides capable of encoding at least one antigenic component of an infectious agent. To facilitate translation of the RNA sequence some embodiments of the described packaged RNA may include translation initiation sequences as described herein.

In some embodiments the RNA sequence incorporated into the VLP will include a retroviral packaging sequence (any sequence from any member of the family Retroviridae, including members of the following subfamilies of Retroviridae—Alpharetrovirus, Betaretrovirus, Gammarretrovirus, Deltaretrovirus, Episilonretrovirus, Lentivirus, Spumavirus). Examples of virus belonging to the retroviridae include Rous Sarcoma Virus (RSV), Human Immuno Deficiency Virus (HIV), Bovine Leukemia Virus among others) that will facilitate inclusion of the RNA into a forming VLP. In some embodiments the retroviral packaging sequence is derived from Rous sarcoma virus, Moloney murine leukemia virus, simian immunodeficiency virus (SIV), HIV, human T-lymphotropic virus, and the like. In a particular embodiment, the retroviral packaging sequence is derived from Rous sarcoma virus. Alternatively, the retroviral packaging sequence may be derived from murine leukemia virus. Examples of suitable components and methods for preparing chimeric VLPs are described in WO2013/148302 and WO 2015/095167.

In addition, the RNA sequences included in the VLP may be capable of encoding nonstructural alphavirus proteins. For example, in some embodiments the packaged RNA may encode one or more Sindbis virus or VEEV nonstructural proteins. In some embodiments the packaged RNA may encode the Sindbis virus or VEEV nonstructural protein NSP1. In some embodiments the packaged RNA may encode the Sindbis virus or VEEV nonstructural protein NSP2. In some embodiments the packaged RNA may encode the Sindbis virus or VEEV nonstructural protein NSP3. In some embodiments the packaged RNA may encode the Sindbis virus or VEEV nonstructural protein NSP4. In some embodiments the packaged RNA may encode the Sindbis virus or VEEV nonstructural proteins NSP1, NSP2, NSP3, and NSP4. The packaged RNA may also include the polynucleotide sequence of a protein of interest. For example, the polynucleotide sequence of interest may encode GFP in some embodiments and serve a detectable marker of viral transduction of a target cell.

The described VLPs may also comprise a viral gag protein to provide a viral core structure to the particle. The gag protein is the core structural protein of retroviruses and, in some instances, is capable of forming enveloped virus cores when expressed in eukaryotic cells. This property makes gag proteins particularly useful in the production of VLPs, because they can form the basic structural aspect of the particle and allow for packaging of RNA associated with a retroviral packaging signal sequence. Those skilled in the relevant art will understand that a gag protein from any retrovirus (i.e. any member of the family Retroviridae) may be used to produce the plasmids and VLPs described herein. In some embodiments the gag protein may be derived from Rous sarcoma virus. In some embodiments the gag protein may be derived from murine leukemia virus. In alternative embodiments the gag protein may be derived from SIV, HIV, human T-lymphotropic virus, or similar retrovirus.

Another component of the VLPs described herein is a protein to mediate fusion between the VLP envelope and a host cell (a fusogenic envelope protein). A class of proteins suitable for this purpose is viral fusion proteins, which facilitate virus infection of cells by allowing an enveloped virus to fuse its membrane with that of a host cell. Many of viral fusion proteins also have known, or suspected, cellular receptor proteins that may allow for targeting of selected cell types, or in cases of more ubiquitous receptors, such as sialic acid for influenza virus, more generalized targeting may be achieved. In some instances, viral fusion proteins may work in conjunction with viral attachment proteins, ligands of cellular receptors, receptors of cellular ligands, or accessory proteins, thus proteins of this sort may also be present on the VLP surface in addition to a viral fusion protein. Alternatively, in some embodiments the described VLPs may have a viral fusion protein from one virus and a viral attachment protein of another virus, a ligand of a cellular receptor, a receptor of a cellular ligand, or an accessory protein to facilitate, or direct, targeting of a VLP to a desired cell type. Similarly, the described VLPs may be produced to have more than one fusion protein in the VLP envelope, as this may facilitate fusion to a select variety of cell types. In some embodiments the VLP surface protein(s) will be a type-I membrane protein, which will allow the extracellular domain of the protein to be oriented extracellularly when present on the cell surface. This will also allow the fusion protein to be correctly oriented following budding of a VLP from a packaging cell. Expression of such proteins in a cell will typically result in the cell surface being coated with the proteins, so that budding of a VLP from any part of the cell membrane will provide the VLP with some amount of the fusion protein on its surface. In certain embodiments, the fusogenic envelope protein can be selected from any of the following: haemagglutinin, Rous sarcoma virus (RSV) fusion protein, an E protein of tick borne encephalitis virus and dengue fever virus, the E1 protein of SFV, baculovirus gp64, and Vesicular stomatitis (Indiana) virus-G (VSV-G) protein, preferably a glycoprotein, or fragment or derivative thereof, more preferably from a RNA virus or a retrovirus, or fragment or derivative thereof, most preferably VSV-G or EnvA, or an alteration of VSV-G. The VLP described herein may be capable of binding to a eukaryotic target cell, preferably a human cell. The binding of the VLP may be specific to a target cell. In some embodiments the VLP described herein is not cytopathic to the target cell.

In some embodiments the VLPs described herein include a vesicular stomatitis virus G protein (VSV-G) to mediate cell fusion. In some embodiments the VLPs described herein include an influenza hemaglutinin protein to mediate cell fusion. In some embodiments the VLPs described herein include an influenza neuraminidase protein to facilitate cell fusion. In some embodiments the VLPs described herein include respiratory syncytial virus fusion protein. In some embodiments the VLPs described herein include the rotavirus VP7 protein. Other such fusion proteins will be apparent to those skilled in the art based on desired tropism or cell target of the associated virus.

The VLPs described herein may comprise an alphavirus replicon, wherein the alphavirus replicon includes a polynucleotide encoding at least one immunogenic antigen of an infectious agent, retroviral gag protein, and heterologous fusogenic envelope protein; wherein the VLP does not contain an alphavirus structural protein gene. In some embodiments the alphavirus replicon of the VLP is derived from Sindbis virus or VEEV. For example, the VLPs described herein may have an alphavirus replicon encoding Sindbis virus or VEEV nonstructural proteins NSP1, NSP2, NSP3, and NSP4. In some embodiments the retroviral packaging signal associated with the packaged RNA in the described VLPs is derived from either Rous sarcoma virus or murine leukemia virus. Based on this description, those skilled in the art will readily understand that the described VLPs may be modified to incorporate aspects of viruses that may facilitate VLP stability, RNA packaging, or cell entry. Such modifications should be understood to be within the scope of the disclosures provided herein.

In certain embodiments, at least one psi (P) element is present upstream of the alphavirus replicon. The psi element can be from any of the viruses belonging to the family Retroviridae. In additional embodiments, the at least one psi (P) element is derived from Rous sarcoma virus (RSV). Alternative Retroviridae psi elements include those from: Barmah Forest virus, Middelburg virus, Semliki Forest Virus, Eastern equine encephalitis virus, Western equine encephalitis virus, and Ndumu virus. In certain embodiments, more than one psi element can be present, and the psi elements may be mixed from different Retroviridae in the same replicon construct.

In certain embodiments, the virus nonstructural proteins NSP1, NSP2, NSP3, and NSP4 are from Sindbis virus or Venezuelan equine encephalitis virus; and, optionally, the retroviral packaging signal is derived from Rous sarcoma virus (RSV).

In certain embodiments, the alphavirus is a Venezuelan equine encephalitis virus.

In certain embodiments, the sa-RNA VLP does not comprise or express a retroviral pol gene.

In certain embodiments, the gag protein is any gag protein from the family Retroviridae.

In certain embodiments, the retroviral gag protein is from Rous sarcoma virus (RSV).

In certain embodiments, the fusogenic envelope protein is VSV-G and is capable of binding to a target immune cell.

In certain embodiments, the fusogenic envelope protein is a glycoprotein, or fragment or derivative thereof. The fusogenic envelope protein is selected from the group consisting of haemagglutinin, Rous sarcoma virus (RSV) fusion protein, an E protein of tick borne encephalitis virus and dengue fever virus, the E1 protein of SFV, baculovirus gp64, and Vesicular stomatitis (Indiana) virus-G (VSV-G) protein, preferably a glycoprotein, or fragment or derivative thereof, more preferably from a RNA virus or a retrovirus, or fragment or derivative thereof, most preferably VSV-G or EnvA, or an alteration of VSV-G. The VLP described herein may be capable of binding to a eukaryotic target cell, preferably a human cell. The binding of the VLP may be specific to a target cell. In some embodiments the VLP described herein is not cytopathic to the target cell.

In certain embodiments, the target immune cell is a dendritic cell. In certain embodiments, the target immune cell can include any of the following cells: dendritic cells, macrophages, and any other antigen presenting cells.

In certain embodiments, the sa-RNA VLP is capable of binding to a target cell of a subject, after which the target cell is capable of expressing the immunogenic antigen of the infectious agent, which expression is capable of inducing an immune response, including a T cell response, against coronavirus in the subject.

In certain embodiments, the immune response including a T cell response is induced by a dosing regimen comprising one or two administrations of the sa-RNA VLP, wherein the dose is sufficient to induce an immune response against coronavirus in a subject.

In certain embodiments, the dosing regimen comprises one administration of the SA-RNA VLP, wherein the one dose administration (i.e. a prime alone) is sufficient to induce an immune response against the coronavirus in a subject. In certain embodiments, the immune response is effective to prevent or lessen COVID-19 infection for at least about 4 months, 5 months, 6 months, 7 months, 8 months, 9 months, 10 months, 11 months, 12 months, 13 months, 14 months, 15 months, 16 months, 17 months, or 18 months following a first vaccination with the sa-RNA VLP vaccine. In certain embodiments, the dosing regimen includes at least one booster administration (e.g. at least one prime plus at least one boost) at about 4 months, 5 months, 6 months, 7 months, 8 months, 9 months, 10 months, 11 months, 12 months, 13 months, 14 months, 15 months, 16 months, 17 months, or 18 months following a first vaccination with the sa-RNA VLP vaccine. In certain embodiments the prime and boost at the same vaccine, and in alternative embodiments the prime and boost are heterologous vaccines.

The VLPs described herein may be produced in a variety of ways (See for example, the methods described in WO2013/148302 and WO 2015/095167), as will be apparent to those skilled in the art based on the provided disclosure. The commonality to these various methods is the expression of the described plasmids in a cell capable of expressing the necessary proteins (gag and a fusion protein) and producing the alphavirus-based RNA replicon. Accordingly, a method of producing a VLP described herein may include co-transforming, transfecting, or nucleofecting a eukaryotic cell with a plasmid comprising a polynucleotide sequence encoding an alphavirus replicon, wherein the alphavirus replicon includes a polynucleotide encoding at least one immunogenic antigen of an infectious agent; a plasmid comprising a polynucleotide sequence encoding a retroviral gag protein; and a plasmid comprising a polynucleotide sequence encoding a heterologous fusogenic envelope protein; and culturing the co-transformed cell under conditions suitable to cause each plasmid to produce its encoded product, thereby producing a virus-like particle. In some embodiments the polynucleotide sequence encoding the alphavirus replicon may be derived from Sindbis virus or VEEV. In some embodiments the alphavirus replicon may have polynucleotide sequences that encode Sindbis virus or VEEV nonstructural proteins NSP1, NSP2, NSP3, NSP4, and a retroviral packaging signal. In some embodiments the retroviral packaging signal may be derived from either Rous sarcoma virus or murine leukemia virus. In some embodiments the polynucleotide sequence encoding the gag protein is derived from Rous sarcoma virus. In some embodiments the polynucleotide sequence encoding the heterologous fusogenic envelope protein encodes VSV-G. The glycoprotein of the vesicular stomatitis virus (VSV-G) is a transmembrane protein that functions as the surface coat of the wild type viral particles. The VSV-G protein target splenocytes, immune cells and in particular antigen presenting cells (APC) or dendritic cells (DC), liver cells including hepatocytes or non-parenchymal cells, and activated or proliferating cardiomyocytes. Thus, in certain preferred embodiments, the fusogenic envelope protein is VSV-G which serves to bind the VLP to desired target cells in a subject or patient, such as dendritic cells or APCs.

### 4.4. Immunogenic Antigens from Infectious Agents Encoded by the saRNA VLP

In certain embodiments, the infectious agent is a virus. In certain embodiments, the infectious agent is a bacterium. Exemplary bacteria include one or more of the pathogenic bacterial species Bacteroides, Clostridium, Streptococcus, Staphylococcus, Pseudomonas, Haemophilus, Legionella, Mycobacterium, Escherichia, Salmonella, Shigella, or Listeria.

In additional embodiments, the virus is a coronavirus. In certain embodiments, the coronavirus is SARS-CoV-2. In certain embodiments, the immunogenic antigen is a surface antigen. In additional embodiments, the sa-RNA VLP is an sa-RNA SARS-CoV-2 VLP vaccine. In certain embodiments, the surface antigen has at least 80% identity to a coronavirus spike protein, coronavirus membrane protein, coronavirus hemagglutinin esterase (HE), coronavirus envelope protein, or any combination thereof. In certain embodiments, the surface antigen has 80% identity to a coronavirus spike protein. Additionally, immunogenic antigens can include surface proteins, lipopolysaccharides, and peptidoglycans on the bacterial cell wall. Fusions or chimeric antigen constructs are also provided. An example of such antigens include pili, lipopolysaccharides, various cell wall components, and flagella (See, Immunology for Pharmacy, 2021 pgs. 147-151 https://doi.org/10.1016/B978-0-323-06947-2.10018-5).

In certain embodiments, the spike protein sequence has at least 90% identity with a coronavirus spike protein or ectodomain amino acids 1-1208 of SEQ ID NO: 1, or a combination thereof. In certain embodiments, the spike protein sequence comprises SEQ ID NO: 1 or the ectodomain region of amino acids 1-1208 of SEQ ID NO: 1, or a combination thereof.

In certain embodiments, the spike protein sequence is encoded by a codon optimized sequence of SEQ ID NO: 1; or a codon optimized ectodomain sequence of amino acids 1-1208 of SEQ ID NO: 1.

SEQ ID NO:17 is the amino acid sequence for CB-107 and comprises:
signal domain (SEQ ID NO: 10)
N-terminus domain (SEQ ID NO: 11)
Receptor Binding Domain (SEQ ID NO: 12)
S2 domain (SEQ ID NO: 13)

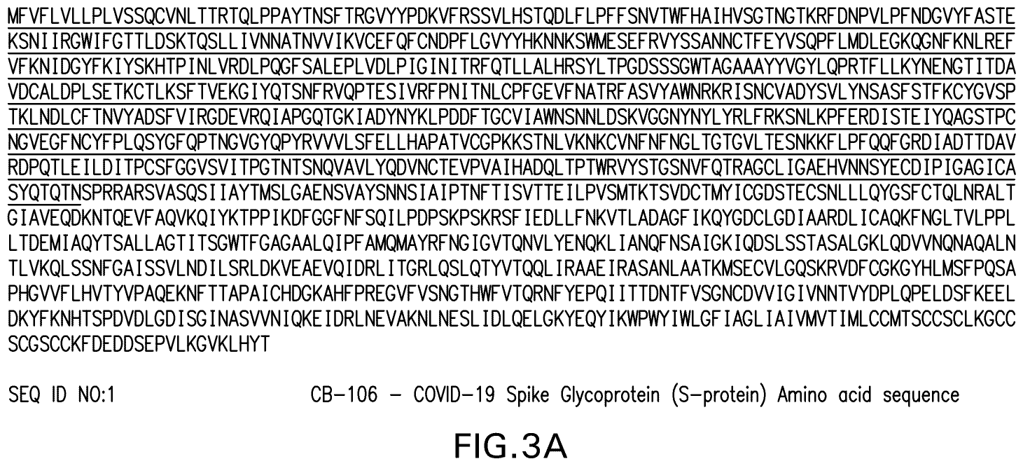

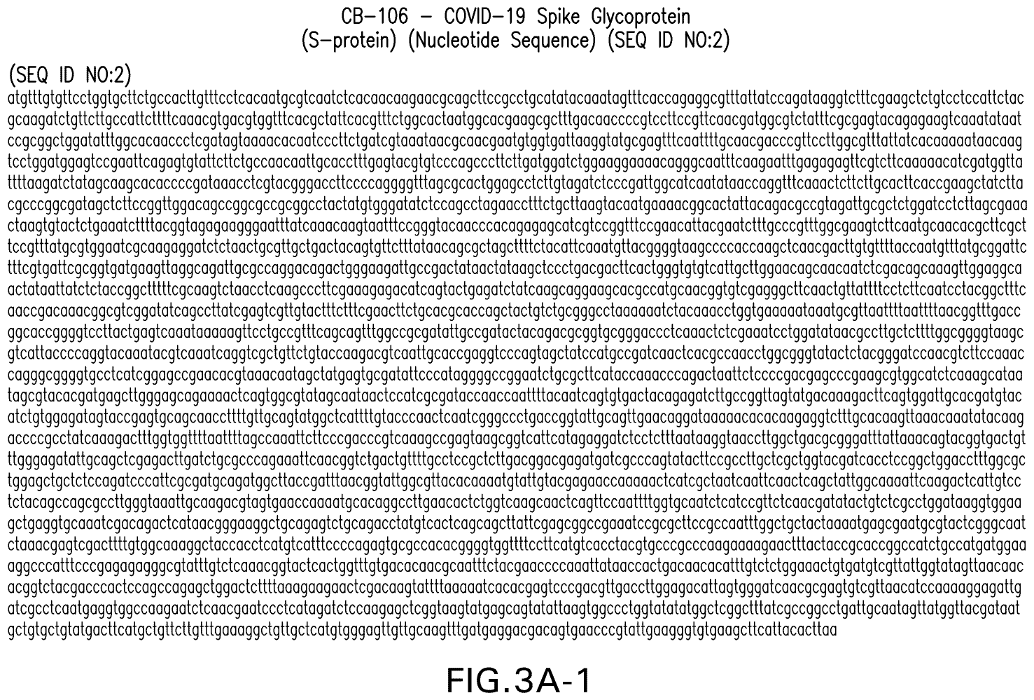

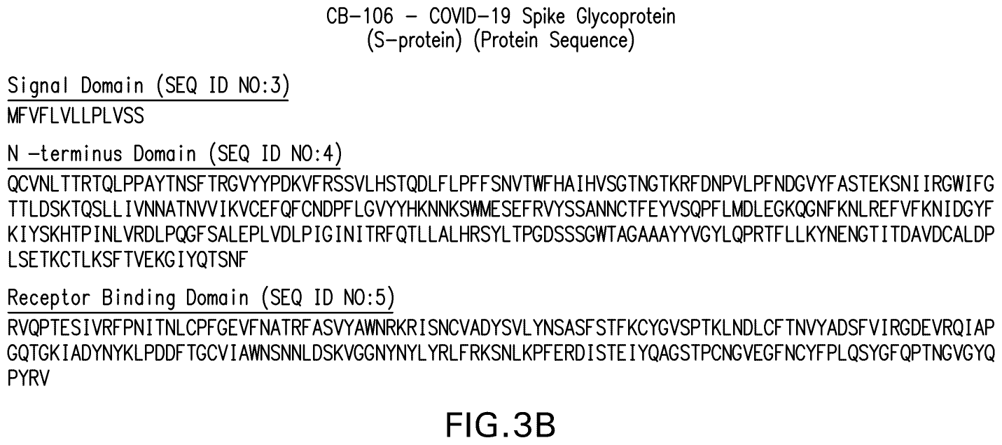

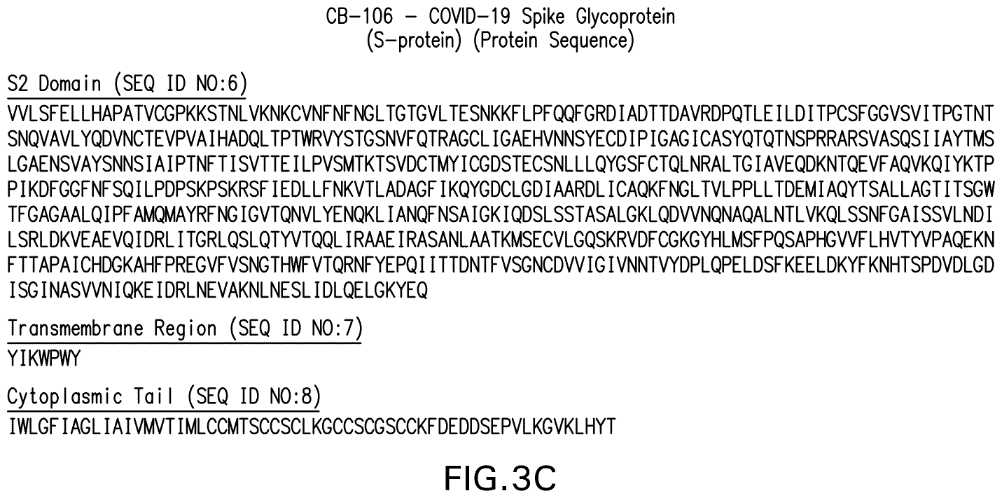

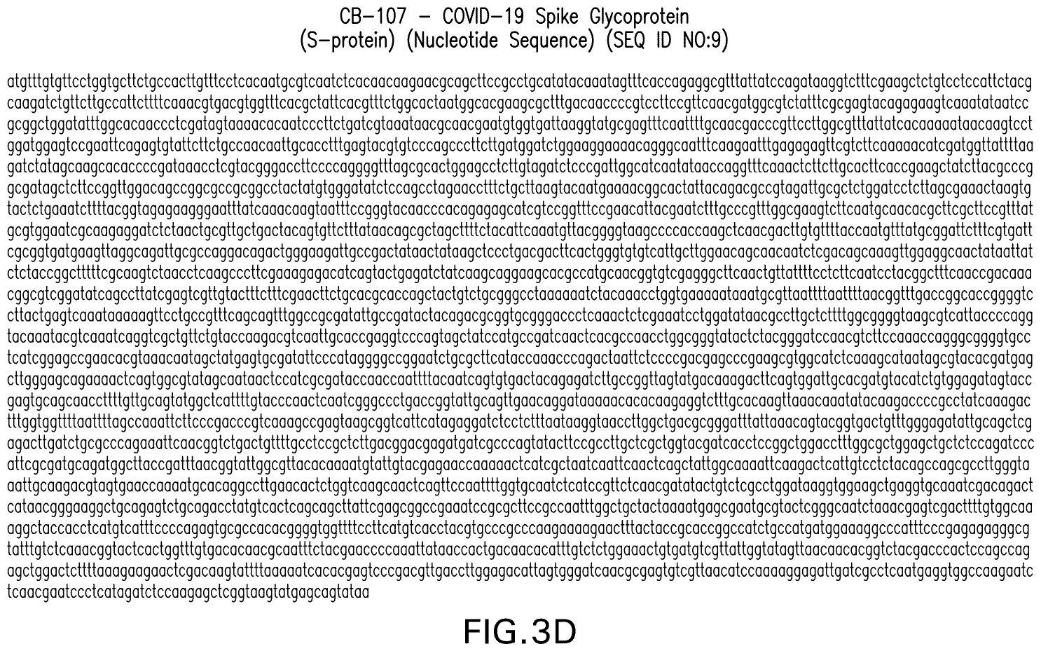

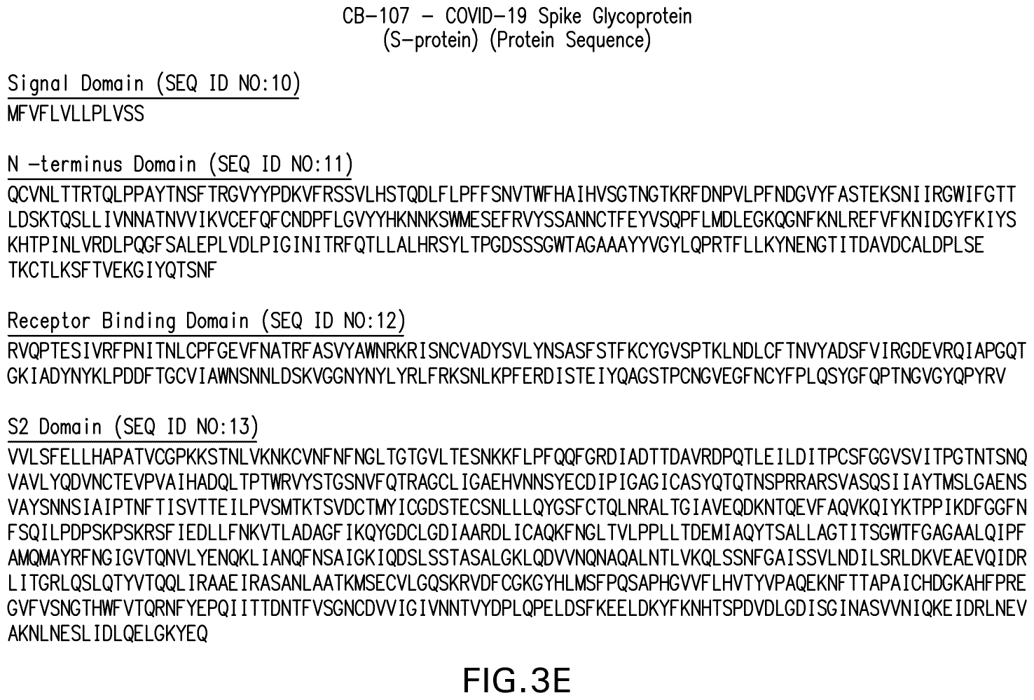

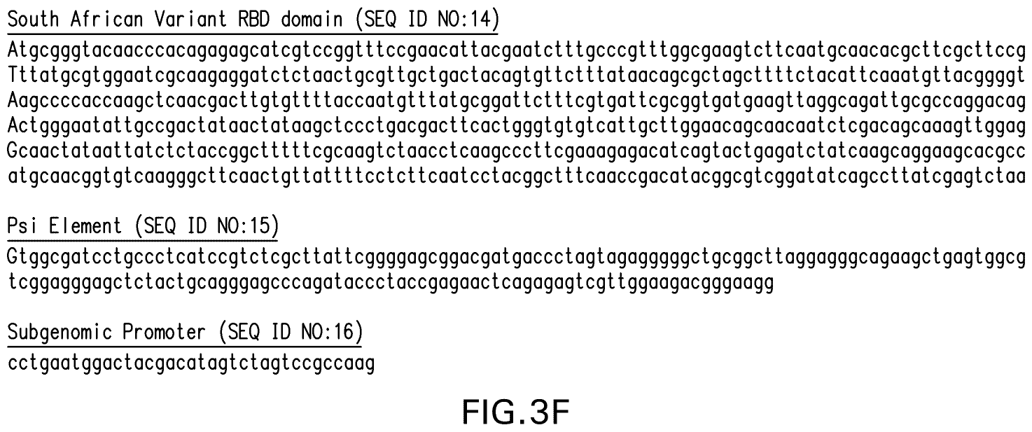

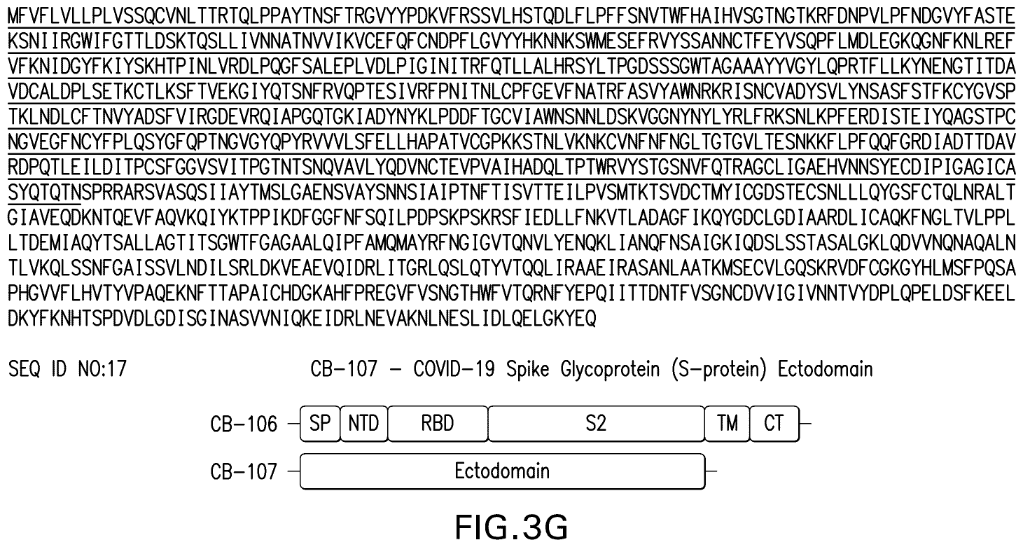

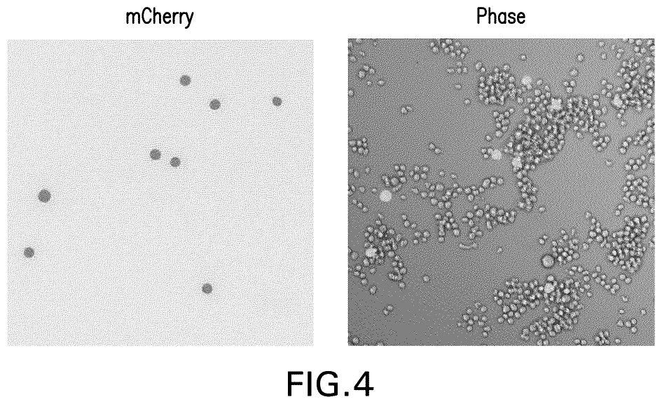

These features are further shown in the lower panel of FIG. 3G illustrating the “overlap” of CB-107 and CB-106 that encodes the full-length spike protein (SEQ ID NO 1). This shows that CB-107/SEQ ID NO:17 is 65 amino acids shorter than SEQ ID NO: 1. The 65 amino acids are the last 65 amino acid residues—these are present in the CB-106/SEQ ID NO: 1 but not CB-107/SEQ ID NO:17. The transmembrane domain and the cytoplasmic tail are the regions not present in CB-107/SEQ ID NO:17.

In certain embodiments, the spike protein sequence comprises a sequence having at least 80% identity with a sequence selected from SEQ ID NOs: 3-8. In certain embodiments, the spike protein sequence comprises a sequence having at least 90% identity with a sequence selected from SEQ ID NO: 3-8. In certain embodiments, the spike protein sequences comprises two or more sequences each having at least 80% sequence identity with a sequence selected from SEQ ID NOs: 3-8. In certain embodiments, the spike protein sequence comprises two or more sequences selected from SEQ ID NOs: 3-8.

In certain embodiments, the spike protein sequence comprise a signal domain, an N-terminus domain, a receptor binding domain (RBD), an S2 domain, a transmembrane region, a cytoplasmic tail, or a combination thereof.

In certain embodiments, the spike protein sequence comprises a sequence having at least 80% identity with a sequence selected from SEQ ID NOs: 10-13. In certain embodiments, the spike protein sequence comprises a sequence having at least 90% identity with a sequence selected from SEQ ID NO: 10-13. In certain embodiments, the spike protein sequences comprises two or more sequences each having at least 80% sequence identity with a sequence selected from SEQ ID NOs: 10-13. In certain embodiments, the spike protein sequence comprises two or more sequences selected from SEQ ID NOs: 10-13.

In certain embodiments, the spike protein sequence is encoded by a sequence having at least 90% identity with a sequence selected from SEQ ID NO: 14. In certain embodiments, the spike protein sequence is encoded by SEQ ID NO: 14.

In certain embodiments, the sa-RNA SARS-CoV-2 VLP vaccine provided herein can prevent or decrease symptoms of the disease COVID-19. Such symptoms which may be lessened or prevented include any one or more of the following: fever, high temperature (>37.3° C.), cough, myalgia, sputum production, headache, haemoptysis, diarrhoea, dyspnoea and in some cases, acute respiratory distress syndrome (ARDS), acute cardiac injury or secondary infection. ARDS, caused by SARS-CoV-2 is often associated with hypoxemia despite relatively normal lung function. The ALI is associated with cellular infiltration of the airways and inflammation. High mobility group box protein 1 (HMGB1) and interleukin-6 (IL-6) are among the pro-inflammatory cytokines implicated in ALI. Additional symptoms which may be prevented include those associated with acute respiratory distress syndrome (ARDS) and/or acute lung injury (ALI) and/or pneumonia.

### 4.5. Targeting Cells with a Self-Amplifying RNA VLP Vaccines to Deliver SARS-CoV-2 Antigens

Coronaviridae is a family of viruses (e.g., MERS-CoV and Severe Acute Respiratory Syndrome (SARS-CoV)) that primarily infect the upper respiratory and gastrointestinal tracts of mammals and birds, and that are responsible for acute and chronic diseases of the respiratory, hepatic, gastrointestinal and neurological systems. Coronaviruses are enveloped positive-sense, single-stranded RNA viruses with a nucleocapsid of helical symmetry and virions with a crown-like appearance. They have the largest genome among all RNA viruses, typically ranging from 27 to 32 kb. The genome is packed inside a helical capsid formed by the nucleocapsid protein (N) and further surrounded by an envelope. Associated with the viral envelope are at least three structural proteins: The membrane protein (M) and the envelope protein (E) are involved in virus assembly, whereas the spike protein (S) mediates virus entry into host cells. Some coronaviruses also encode an envelope-associated hemagglutinin-esterase protein (HE). Among these structural proteins, the spike forms large protrusions from the virus surface, giving coronaviruses the appearance of having crowns (hence their name; corona in Latin means crown). In addition to mediating virus entry, the spike is an important determinant of viral host range and tissue tropism and a major inducer of host immune responses.

The virions of each coronavirus are approximately 100 nm with a crown-like appearance because of the club-shaped spike (S) proteins projecting from the surface of the envelope. The spike protein is the viral membrane protein that is responsible for cell entry and includes an S1 domain, which is responsible for binding the cell surface receptor, and an S2 domain, which is a membrane-anchored subunit.

Upon entering an infected cell, coronaviruses transcribe their RNA and the viruses replicate in the cytoplasm of the infected cell. Replication is mediated by the synthesis of an antisense RNA strand, which is provided as a template for additional viral genomes and transcription. The viruses then assemble and are released from the infected cell.

The amino acid sequence of the full length COVID-19 spike glycoprotein (S-protein) can be found at: Genbank ID 43740568. Underlined is the S1 domain.

SEQ ID NO: 1
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLFNKVTLADAGFIKQYGDCLGDIAARDLICAQKE
NGLTVLPPLLTDEMIAQYTSALLAGTITSGWTFGAGAALQIPFAM
QMAYRENGIGVTQNVLYENQKLIANQFNSAIGKIQDSLSSTASAL
GKLQDVVNQNAQALNTLVKQLSSNFGAISSVLNDILSRLDKVEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFDEDD
SEPVLKGVKLHYT

The full-length ectodomain is from residues 1 to 1208 of the complete SARS CoV S protein and corresponds to SEQ ID NO:17, also shown in FIG. 3G).

### 4.6. Pharmaceutical Compositions

Additional aspects provided herein include a pharmaceutical composition comprising any of the sa-RNA SA-RNA VLP s described herein.

In certain embodiments, the composition further comprises a pharmaceutically acceptable carrier, diluent, and/or additive, or any combination thereof.

In certain embodiments, the pharmaceutical composition is capable of inducing an immune response against coronavirus in a mammalian subject.

In certain embodiments, wherein following administration of the composition to the subject, the sa-RNA VLP is capable of producing or inducing a T cell response against coronavirus in a mammalian subject.

In certain embodiments, the coronavirus is SARS-CoV-2 and the sa-RNA VLP is sa-RNA SARS-CoV-2 VLP.

In certain embodiments, the sa-RNA VLP or pharmaceutical composition described herein is for use in diminishing or preventing at least one symptom of a coronavirus infection in a mammalian subject.

In certain embodiments, the diminishing or preventing comprises inducing coronavirus-specific immunity against SARS-CoV-2 (COVID-19).

In certain embodiments, the sa-RNA VLP or pharmaceutical composition described herein are for use in inducing cellular and or humoral immunity in a mammalian subject.

In certain embodiments, the sa-RNA VLP or pharmaceutical composition described herein is for use in eliciting an immune response in a mammalian subject.

In certain embodiments, said inducing or eliciting an immune response is an immune response against SARS-CoV-2 (COVID-19).

In certain embodiments, the sa-RNA VLP or pharmaceutical compositions described herein are for use in inducing neutralizing antibodies against SARS-CoV-2 in a mammalian subject.

### 4.7. Method of Diminishing or Preventing A Coronavirus Infection

Additional aspects provided herein include a method of diminishing or preventing a viral or bacterial infection in a mammalian subject comprising administering any of the sa-RNA VLP's, or any of the pharmaceutical compositions described herein to the subject.

Additional aspects provided herein include a method of diminishing or preventing a coronavirus infection in a mammalian subject comprising administering any of the sa-RNA VLP's, or any of the pharmaceutical compositions described herein to the subject.

Additional aspects provided herein include a method of inducing cellular and or humoral immunity against a coronavirus in a mammalian subject, comprising administering any of the sa-RNA VLP's, or any of the pharmaceutical compositions described herein to the subject.

Additional aspects provided herein include a method of eliciting an immune response against a coronavirus in a mammalian subject, comprising administering any of the sa-RNA VLP's, or any of the pharmaceutical compositions described herein to the subject.

In some embodiments, methods for increasing an immune response, or eliciting a desired immune response in a patient in need thereof are provided. In some embodiments, the methods comprise administering an effective amount of an sa-RNA VLP's described herein, to the patient.

Diseases that the compositions and methods described herein can diminish or prevent microbial infections such as a viral infection, yeast infection, fungal infection, protozoan infection and/or bacterial infection.

Exemplary bacterial infections include those caused by one or more of the pathogenic bacterial species Bacteroides, Clostridium, Streptococcus, Staphylococcus, Pseudomonas, Haemophilus, Legionella, Mycobacterium, Escherichia, Salmonella, Shigella, or Listeria.

By a “viral infection” is meant an infection caused by the presence of a virus in the body. Viral infections include chronic or persistent viral infections, which are viral infections that are able to infect a host and reproduce within the cells of a host over a prolonged period of time-usually weeks, months or years, before proving fatal. Viruses giving rise to chronic infections that which may be treated in accordance with the present invention include, for example, the human papilloma viruses (HPV), Herpes simplex, and other herpes viruses, the viruses of hepatitis B and C as well as other hepatitis viruses, human immunodeficiency virus, and the measles virus, all of which can produce important clinical diseases. Prolonged infection may ultimately lead to the induction of disease which may be, e.g., in the case of hepatitis C virus liver cancer, fatal to the patient. Other chronic viral infections which may be treated in accordance with the present invention include Epstein Barr virus (EBV), as well as other viruses such as those which may be associated with tumors.

Examples of viral infections which can be prevented or whose symptoms can be diminished by vaccination with the sa-RNA based VLP compositions and methods described herein include, but are limited to, viral infections caused by retroviruses (e.g., human T-cell lymphotrophic virus (HTLV) types I and II and human immunodeficiency virus (HIV)), herpes viruses (e.g., herpes simplex virus (HSV) types I and II, Epstein-Ban virus and cytomegalovirus), arenaviruses (e.g., lassa fever virus), paramyxoviruses (e.g., morbillivirus virus, human respiratory syncytial virus, and pneumovirus), adenoviruses, bunyaviruses (e.g., hantavirus), coronaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., Sendai virus and influenza viruses A, B and C), papovaviruses (e.g., papillomaviruses), picornaviruses (e.g., rhinoviruses, enteroviruses and hepatitis A viruses), poxviruses, reoviruses (e.g., rotaviruses), togaviruses (e.g., rubella virus), and rhabdoviruses (e.g., rabies virus). The treatment and/or prevention of a viral infection includes, but is not limited to, preventing or alleviating one or more symptoms associated with said infection, the inhibition, reduction or suppression of viral replication and/or viral load, and/or the enhancement of the immune response.

In some embodiments, methods of inhibiting a viral or bacterial replication or reproduction in a subject having a viral or bacterial infection are provided. In some embodiments, the methods comprise administering to the subject with the viral or bacterial infection an effective amount of sa-RNA based VLP compositions to the patient. In some embodiments the viral or bacterial load in the patient is reduced or is undetectable. The viral or bacterial load can be reduced to undetectable levels or by at least 10, 20, 30, 40, 50, 60, 70, 80, 90, 91, 92, 93, 94, 95, 96, 96, 97, 98, or 99% as compared to the pre-treatment levels.

Additional aspects provided herein include a method of diminishing or preventing a coronavirus infection in a mammalian subject comprising administering the sa-RNA VLP described herein to the subject.

Additional aspects provided herein include a method of inducing cellular and or humoral immunity in a mammalian subject, comprising administering any of the sa-RNA VLP's described herein to the subject.

Additional aspects provided herein include a method of eliciting an immune response in a subject, comprising administering any of the sa-RNA VLP's described herein to the subject.

Additional aspects provided herein include a method of inducing neutralizing antibodies against a coronavirus in a subject, comprising administering any of the sa-RNA VLP's described herein to the subject.

In certain embodiments, the method further includes inducing a T cell response against the coronavirus. In certain embodiments, the T cell response is induced by a regimen comprising one or at least two administrations. In certain embodiments, the coronavirus is SARS-CoV-2 (COVID-19).

Additional aspects provided herein include a method of expressing a heterologous nucleic acid sequence in the host, comprising administering to a subject any of the sa-RNA VLP's described herein, wherein the SA-RNA VLP is capable of binding to a host cell, after which the host cell is capable of expressing the immunogenic antigen of the infectious agent, which expression is capable of inducing a cellular and/or humoral immune response to SARS-CoV-2 in the subject.

Additional aspects provided herein include a method of inducing neutralizing antibodies against SARS-CoV-2 in a mammalian subject, comprising administering any of the sa-RNASA-RNA VLP's, or any of the pharmaceutical compositions described herein to the subject.

In certain embodiments, the method further includes inducing a T cell response against the coronavirus.

In certain embodiments, the immune response is induced by a regimen comprising one or two administrations of any of the sa-RNA VLP or pharmaceutical compositions described herein.

In certain embodiments, the immune response is induced by a regimen comprising one administration of any of the sa-RNA VLP s or pharmaceutical compositions described herein.

In certain embodiments, the coronavirus is SARS-CoV-2.

Additional aspects provided herein include a method of expressing a recombinant polynucleotide encoding an immunogenic antigen of an infectious agent in a subject, comprising administering any of the sa-RNA VLP s described herein, or any of the pharmaceutical compositions described herein to the subject.

In certain embodiments, the sa-RNA VLP is capable of binding to a target cell, after which the target cell is capable of expressing the immunogenic antigen of the infectious agent, which expression is capable of inducing a cellular and/or humoral immune response to SARS-CoV-2 in the subject.

### 4.8. sa-RNA VLP Composition Stability

Additional aspects provided herein include wherein any of the sa-RNA VLP or compositions thereof is stable at from the range of about 4° C.-10° C. for at least about one-six months. In certain embodiments, the sa-RNA VLP is stable for at least about six to nine months. In certain embodiments, the sa-RNA VLP is stable for at least about nine to twelve months. Additional embodiments include wherein the sa-RNA VLP is stable for 2° C.-8° C. for at least one-six months. Additionally, in certain embodiments, the sa-RNA VLP is stable at about −80° C. for at least 2 years.

### 4.9. Production of sa-RNA VLPs

Additional aspects provided herein include a method of producing any of the sa-RNA VLPs described herein, comprising:
a. co-transforming a eukaryotic cell with:
   a first vector comprising a polynucleotide sequence encoding the alphavirus replicon;
   a second vector comprising a polynucleotide sequence encoding a retroviral gag protein; and
   a third vector comprising a polynucleotide sequence encoding the fusogenic envelope protein;
b. culturing the co-transformed eukaryotic cell under conditions suitable to cause each vector to produce its encoded product, thereby producing the sa-RNA VLP; and
c. isolating the sa-RNA VLP from the eukaryotic cell.

Additional aspects provided herein include an sa-RNA VLP produced by any of the methods described herein.

Described herein are plasmids for use in producing VLPs carrying an alphavirus-based replicon that does not encode alphavirus structural proteins. To produce VLPS of this sort, several components may be produced by transfecting or nucleofecting one or more plasmids encoding these components into a cell line for in vitro production. In some embodiments, these components are encoded by separate plasmids to reduce the likelihood that the resulting VLP will be replication competent. For example, a multi-plasmid system may be used where one plasmid encodes the genetic material, such as an RNA polynucleotide encoding bacterial or viral antigenic elements, such as at least one SARS-COVID-2 spike protein, or antigenic fragment thereof to be packaged by the VLP; another encodes the structural proteins of the VLP, such as gag; and another plasmid encodes a fusion protein, such as VSV-G, to facilitate fusion of the VLP to the membrane of a target cell.

The plasmids encoding the genetic material to be packaged by a host cell can take a variety of forms, such as selectable or inducible plasmids, but generally have some common characteristics. For example, plasmids encoding an RNA alphavirus-based replicon described herein may include a promoter sequence, a retroviral packaging signal sequence, translation initiation sequences, nonstructural alphavirus proteins, a cloning site for inserting a gene or polynucleotide of interest, an inserted gene or polynucleotide of interest, a 3′ untranslated region, and a poly-adenosine tail segment.

In some embodiments the described plasmids include a promoter element that allows for segments of the plasmid to be transcribed by a host cell. In some embodiments the plasmid sequence may be transcribed into RNA to be packaged into a VLP. In most embodiments of the described plasmids, the promoter sequence will be located upstream of all of the translatable elements included within the plasmid. In some embodiments described herein the promoter sequence will be derived from a virus, such as cytomegalovirus (CMV), or simian virus 40 (SV40). Numerous other promoter sequences are well known to those skilled in the art and their use with the plasmids described herein would be apparent based on the description provided.

In some embodiments the described alphavirus replicons or plasmids encoding the genetic material to be packaged by a host cell into the VLP can include a polynucleotide sequence that encodes a retroviral packaging signal sequence (also known as a psi (Ψ) element) to allow one or two copies of the RNA sequence transcribed from the plasmid to be packaged into a VLP particle formed in a host cell. In the context of an alphavirus replicon, In some embodiments the plasmids described herein include a polynucleotide sequence that encodes a retroviral packaging sequence derived from Rous sarcoma virus, Moloney murine leukemia virus, simian immunodeficiency virus (SIV), HIV, human T-lymphotropic virus, and the like. In a particular embodiment, the retroviral packaging sequence is derived from Rous sarcoma virus. Alternatively, the retroviral packaging sequence is derived from murine leukemia virus.

Another aspect of the plasmids encoding the genetic material to be packaged by a host cell described herein are translation initiation sequences, which allow the RNA sequence encoded by the plasmid to be translated in a host cell. In some embodiments the described translation initiation sequences may be capable of allowing for expression of alphavirus-based nonstructural proteins, which can replicate the RNA sequence carried by the described VLPs once it is delivered to the host cell. In some embodiments, the described translation initiation sequences may be capable of allowing for expression of a gene of interest. In some embodiments the translation initiation sequence may allow for the gene of interest to be translated by host cell translation complexes. In some embodiments the translation initiation sequences described herein may be derived from an alphavirus, such as Sindbis virus or VEEV. In other embodiments the translation initiation sequences may be derived from other genes, such as virus genes known to have translation initiation sequences capable of initiating translation of an RNA sequence by mammalian translation complexes. Alternatively, the translation initiation sequences may be derived from other genes, such as the native translation initiation sequence of the gene of interest inserted into the described alphavirus replicon. In some embodiments the translation initiation sequences described herein may be located at more than one location in the packaged RNA molecule, and thus may be encoded one or more times by the described plasmids. For example, it may be desirable to translate the described Sindbis or VEEV nonstructural proteins separately from a gene of interest encoded by the package RNA molecule. In such an instance, both the polynucleotide(s) encoding the nonstructural proteins and the polynucleotide encoding the protein of interest will have separate translation initiation sequences located 5′ of their position in the plasmid and packaged RNA. Based on this description, those skilled in the art will understand that a variety of translation initiation sequences capable of promoting the translation of RNA in a mammalian cell may be incorporated to the described VLP-packaged RNAs described herein.

The plasmids encoding genetic material to be packaged by a host cell may also include polynucleotides that encode nonstructural alphavirus proteins, such as nonstructural proteins from Sindbis virus or VEEV. For example, in some embodiments the described plasmids may include polynucleotides that encode one or more Sindbis virus nonstructural proteins. In some embodiments the described plasmids may include polynucleotides that encode one or more VEEV nonstructural proteins. In some embodiments described plasmids may include polynucleotides that encode the Sindbis virus or VEEV nonstructural protein NSP1. In some embodiments described plasmids may include polynucleotides that encode the Sindbis virus or VEEV nonstructural protein NSP2. In some embodiments described plasmids may include polynucleotides that encode the Sindbis virus or VEEV nonstructural protein NSP3. In some embodiments described plasmids may include polynucleotides that encode the Sindbis virus or VEEV nonstructural protein NSP4. In some embodiments described plasmids may include polynucleotides that encode the Sindbis virus or VEEV nonstructural proteins NSP1, NSP2, NSP3, and NSP4. In some embodiments the polynucleotide of the described plasmid that encodes the alphavirus nonstructural proteins will be derived from the corresponding genomic sequence of an alphavirus genome, such as that of Sindbis virus or VEEV. In some embodiments, the polynucleotides encoding the alphavirus nonstructural proteins are void of any polynucleotides that encode the alphavirus structural proteins, regardless of whether the structural proteins are from the same or a different alphavirus than the nonstructural protein sequences present.

In some embodiments the described plasmids may encode an RNA polynucleotide sequence to be packaged into a VLP, which can then be delivered to a host cell by VLP-mediated transduction of the cell. Once the RNA polynucleotide sequence has been delivered to the target cell a polynucleotide of interest encoded by the RNA may provide for expression of a protein of interest. Accordingly, the plasmids described herein are designed to encode an RNA for packaging into a VLP that can express a gene of interest once inside a target cell. Therefore, in some embodiments the described plasmids will include a polynucleotide sequence of interest. In some embodiments of the described plasmid, the polynucleotide sequence of interest may encode a protein of interest. For example, the polynucleotide sequence of interest may encode GFP in some embodiments and serve a detectable marker of viral transduction of a target cell.

Another plasmid useful in the production of the VLPs described herein is a plasmid that encodes a virus structural protein. One such class of proteins is the retroviral group-specific antigen (gag) protein. The gag protein is the core structural protein of retroviruses and, in some instances, is capable of forming enveloped virus cores when expressed in eukaryotic cells. This property makes gag proteins particularly useful in the production of VLPs, because they can form the basic structural aspect of the particle and allow for packaging of RNA associated with a retroviral packaging signal sequence. Accordingly, described herein are plasmids that include a polynucleotide that encodes a retroviral gag protein. In some embodiments, the described plasmids include a polynucleotide that encodes a retroviral gag protein and a promoter polynucleotide sequence that allows for the gag gene sequence to be transcribed into mRNA by host cell RNA polymerase. In one embodiment, the promoter polynucleotide sequence is derived from a virus, such as SV40 or CMV. In some embodiments, the plasmid will further include a polynucleotide that encodes a protein of interest. Those skilled in the relevant art will understand that a polynucleotide sequence of a gag protein from any retrovirus may be used to produce the plasmids and VLPs described herein. In some embodiments the polynucleotide sequence encoding the gag protein may be derived from Rous sarcoma virus. In some embodiments the polynucleotide sequence encoding the gag protein may be derived from murine leukemia virus. In some embodiments the polynucleotide sequence encoding the gag protein may be derived from SIV. In some embodiments the polynucleotide sequence encoding the gag protein may be derived from human T-lymphotropic virus.

Another plasmid useful in the production of the VLPs described herein is a plasmid that encodes a protein to mediate fusion between the VLP envelope and a host cell. A class of proteins suitable for this purpose is viral fusion proteins, which facilitate virus infection of cells by allowing an enveloped virus to fuse its membrane with that of a host cell. Many of viral fusion proteins also have known, or suspected, cellular receptor proteins that may allow for targeting of selected cell types, or in cases of more ubiquitous receptors, such as sialic acid for influenza virus, more generalized targeting may be desired. In some instances, viral fusion proteins work in conjunction with viral attachment proteins, ligands for cellular receptor, a receptor for a cell ligand, or accessory proteins, thus proteins of this sort may also be encoded by the described plasmids, in addition to, or also by, the plasmid encoding a viral fusion protein. Alternatively, in some embodiments a viral fusion protein from one virus may be encoded by the described plasmid along with a viral attachment protein of another virus, a ligand of a cellular receptor, a receptor of a cell ligand, or an accessory protein to facilitate, or direct, targeting of a VLP to a desired cell type. In some embodiments the viral fusion protein, viral attachment protein, ligand of a cellular receptor, receptor of a cell ligand, or accessory protein will be a type-I membrane protein, which will allow the extracellular domain of the protein to be oriented extracellularly when present on the cell surface. This will also allow the fusion protein to be correctly oriented following budding of a VLP from a packaging cell. Expression of such proteins in a cell will typically result in the cell surface being coated with the proteins, so that budding of a VLP from any part of the cell membrane will provide the VLP with some amount of the protein(s) on its surface. In some embodiments, the described plasmids include a polynucleotide that encodes a viral fusion protein and a promoter polynucleotide sequence that allows for the fusion protein gene sequence to be translated into mRNA by host cell RNA polymerase. In one embodiment, the promoter polynucleotide sequence is derived from a virus, such as SV40 or CMV. In some embodiments, the described plasmids include a polynucleotide that encodes a viral attachment protein and a promoter polynucleotide sequence that allows for the attachment protein gene sequence to be translated into mRNA by host cell RNA polymerase. In one embodiment, the promoter polynucleotide sequence is derived from a virus, such as SV40 or CMV. In some embodiments the plasmids described herein include a polynucleotide that encodes a vesicular stomatitis virus G protein. In some embodiments the plasmids described herein include a polynucleotide that encodes the influenza hemagglutinin protein. In some embodiments the plasmids described herein include a polynucleotide that encodes the influenza neuraminidase protein. In some embodiments the plasmids described herein include a polynucleotide that encodes the respiratory syncytial virus fusion protein. In some embodiments the plasmids described herein include a polynucleotide that encodes the rotavirus VP7 protein. Other such fusion proteins will be apparent to those skilled in the art based on desired tropism or cell target of the associated virus.

Provided herein are cells comprising the plasmids described to produce VLPs. These cells may be used to produce the VLPs described herein by transcribing or expressing the polynucleotides of the plasmids. For instance, a mammalian cell transfected with a plasmid having a polynucleotide sequence encoding an alphavirus RNA construct having a gene or polynucleotide of interest and a packaging signal, a plasmid encoding a retroviral gag protein, and a plasmid encoding a viral fusion protein could produce a VLP having the expressed viral fusion protein on its surface with one or two copies of the encoded alphavirus RNA construct housed inside the VLP. Furthermore, because none of these plasmids encode alphavirus structural proteins the possibility of creating an infectious virus is substantially reduced compared to systems that do include alphavirus structural proteins.

The described cells may be any eukaryotic cell capable of transcribing, and where necessary (such as in the case of the gag and fusion proteins), translating the polynucleotides of the described plasmids. The cells will likely be mammalian cells in many embodiments. For example, rodent cells, such as murine, hamster (CHO or BHK-21), or rat cells could be used to express the described plasmids; canine cells, such as Madin Darby canine kidney cells, could be used to express the described plasmids; primate cells, such as vero cells, could be used to express the described plasmids; and human cells, such as HEK293T cells (human kidney), Hep-2 cells (human airway), Caco-2 (intestine), HeLa (epithelium), and other such cell lines known in the art, could be used to express the described plasmids. In some embodiments the described cells can be transfected and selected, using standard transfection and selection methods known in the art, to stably comprise one or more of the described plasmids.

In some embodiments the cell lines described herein will contain a plasmid comprising a polynucleotide sequence encoding an alphavirus replicon wherein the alphavirus replicon encodes a protein of interest, a plasmid comprising a polynucleotide sequence encoding a gag protein, and a plasmid comprising a polynucleotide sequence encoding a heterologous fusogenic envelope protein, wherein neither the plasmids nor the cell contain a gene encoding an alphavirus structural protein. In some embodiments the alphavirus replicon may be derived from Sindbis virus or VEEV. In some embodiments the alphavirus replicon may have polynucleotide sequences that encode Sindbis virus or VEEV nonstructural proteins NSP1, NSP2, NSP3, NSP4, and a retroviral packaging signal. In some embodiments the retroviral packaging signal may be derived from either Rous sarcoma virus or murine leukemia virus. In some embodiments the polynucleotide sequence encoding the gag protein is derived from Rous sarcoma virus. In some embodiments the polynucleotide sequence encoding the heterologous fusogenic envelope protein encodes VSV-G.

### 4.10. Pharmaceutical Compositions

Described herein are compositions comprising at least one described VLP and a pharmaceutically acceptable carrier. Such compositions are useful, for example, for administration to subjects in need of expression of an exogenous protein or increased expression of a protein normally found in those of the same species as the subject. The compositions may be formulated as any of various preparations that are known and suitable in the art, including those described and exemplified herein. In some embodiments, the compositions are aqueous formulations. Aqueous solutions may be prepared by admixing the VLPs in water or suitable physiologic buffer, and optionally adding suitable colorants, flavors, preservatives, stabilizing and thickening agents and the like as desired. Aqueous suspensions may also be made by dispersing the VLPs in water or physiologic buffer with viscous material, such as natural or synthetic gums, resins, methylcellulose, sodium carboxymethylcellulose, and other well-known suspending agents.

The compositions may be formulated for injection into a subject. For injection, the compositions described may be formulated in aqueous solutions such as water or in physiologically compatible buffers such as Hanks's solution, Ringer's solution, or physiological saline buffer. The solution may contain one or more formulatory agents such as suspending, stabilizing or dispersing agents. Injection formulations may also be prepared as solid form preparations which are intended to be converted, shortly before use, to liquid form preparations suitable for injection, for example, by constitution with a suitable vehicle, such as sterile water, saline solution, or alcohol, before use.

The compositions may be formulated for aerosolized delivery to a subject. For aerosol delivery, the compositions described may be formulated in aqueous solutions such as water or in physiologically compatible buffers such as Hanks's solution, Ringer's solution, or physiological saline buffer. The solution may contain one or more formulatory agents such as suspending, stabilizing or dispersing agents.

The compositions may be formulated in sustained release vehicles or depot preparations. Such long-acting formulations may be administered by implantation (for example subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well-known examples of delivery vehicles suitable for use as carriers for hydrophobic drugs.

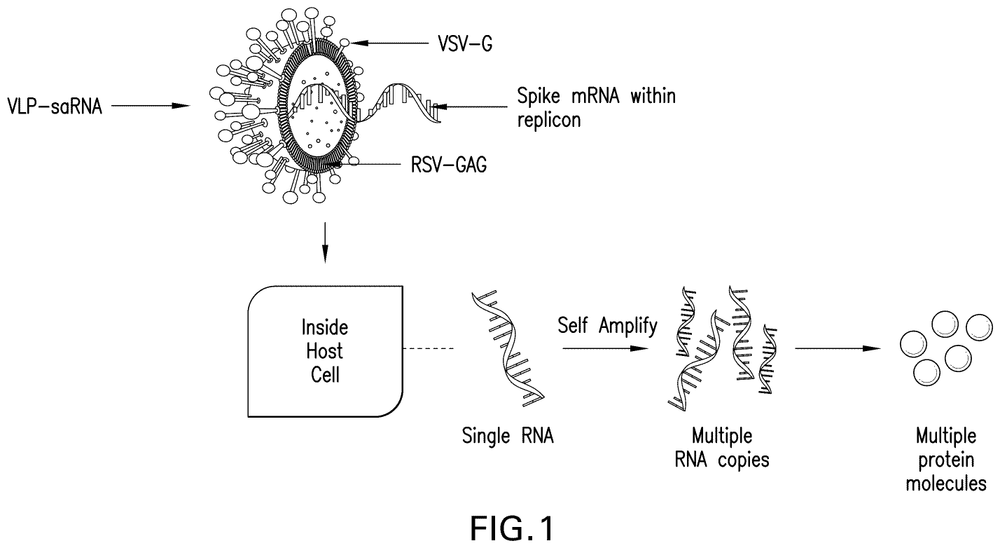

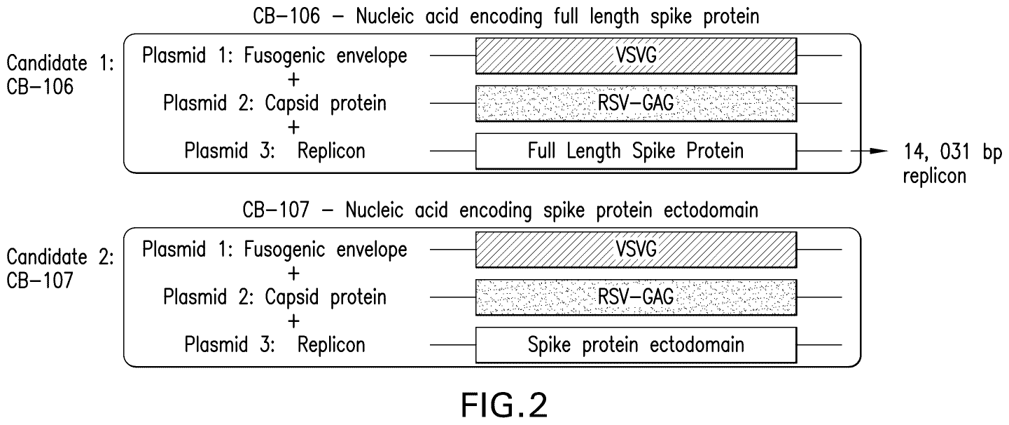

Coronaviruses are large, enveloped, positive-stranded RNA viruses. They have the largest genome among all RNA viruses, typically ranging from 27 to 32 kb. The genome is packed inside a helical capsid formed by the nucleocapsid protein (N) and further surrounded by an envelope. Associated with the viral envelope are at least three structural proteins: The membrane protein (M) and the envelope protein (E) are involved in virus assembly, whereas the spike protein (S) mediates virus entry into host cells. Some coronaviruses also encode an envelope-associated hemagglutinin-esterase protein (HE). Among these structural proteins, the spike forms large protrusions from the virus surface, giving coronaviruses the appearance of having crowns (hence their name; corona in Latin means crown) (FIGS. 1B and 2A). In addition to mediating virus entry, the spike is an important determinant of viral host range and tissue tropism and a major inducer of host immune responses.

In certain aspects, provided herein is a recombinant alphavirus VLP particles, wherein the alphavirus replicon encodes at least one SARS-CoV-2 S glycoprotein (NCBI Reference Sequence: NC_045512.2; Protein_ID: YP_009724390.1; SEQ ID NO: 1) or fragment or derivative thereof (e.g. SEQ ID NOs: 3-8 or 17). See FIGS. 1; FIG. 2; and FIGS. 3A-3C and G. The fragment may be derived from any of the known regions of SARS-CoV-2 S glycoprotein, such as S1, S2, or the ectodomain, or the RBD, or combinations thereof (see Walls et al., Cell, published online Mar. 9, 2020; available at doi.org/10.1016/j.cell.2020.02.058).

In additional embodiments, immunogenic antigens can include surface proteins, lipopolysaccharides, and peptidoglycans from a bacterial cell wall. Fusions or chimeric antigen constructs are also provided. An example of such antigens include pili, lipopolysaccharides, various cell wall components, and flagella (See, Immunology for Pharmacy, 2021 pgs 147-151 https://doi.org/10.1016/B978-0-323-06947-2.10018-5).

### 4.11. Coronavirus Variants

In some embodiments, the virus is a coronavirus. In certain embodiments, the virus is any one or combination of the following coronaviruses: coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2 (COVID-19). In a specific embodiment, the virus is SARS-CoV-2. In particular embodiments, the patient has severe acute respiratory syndrome (SARS). In particular embodiments, the patient has middle eastern respiratory syndrome (MERS). In particular embodiments, the patient has coronavirus disease 2019 (COVID-19).

The percentage amino acid identity of the coronavirus spike and nucleocapsid proteins to SARS-CoV-2 Proteins are shown in Table1 (See, Okba et al. Emerging Infectious Diseases (2020) 26:7 1478-1488).

TABLE 1

| | | Nucleo- | | S1 (N- | S2 (C- |
|---|---|---|---|---|---|
| Virus Type | Virus | capsid | S | terminal) | terminal) |
| Betacoronavirus | SARS-CoV | 90 | 77 | 66 | 90 |
| | MERS- | 49 | 33 | 24 | 43 |
| | CoV | | | | |
| | HCoV- | 34 | 33 | 25 | 42 |
| | OC43 | | | | |
| | HCoV- | 34 | 32 | 25 | 40 |
| | HKU1 | | | | |
| Alphacoronavirus | HCoV- | 28 | 30 | 24 | 35 |
| | 229E | | | | |
| | HCoV- | 29 | 28 | 21 | 36 |
| | NJ63 | | | | |

The VLP can encapsulate RNA encoding different surface epitopes to elicit an immune response. The coronavirus has immunogenic antigens/polypeptides including at least: envelope proteins, membrane proteins, spike proteins, and hemagglutinin. The immunogenic antigen can be a sequence that is at least 7000 homologous or exhibits at least 7000 identity to any of these polypeptides, fragments thereof, or epitopes from a coronavirus. In preferred embodiments, the virus is SARS-CoV-2.

Multiple variants of SARS-CoV-2 have arisen throughout the world in the eighteen months since the virus was first detected. Three mutations in particular, B.1.1.7, B.1.351, and P.1, have become dominant and have increased the effectiveness of the virus. The N501Y mutation, which is in the receptor-binding-domain of the spike protein. The 501Y.V2 and P.1 variants both have two additional receptor-binding-domain mutations, K417N/T and E484K. These mutations increase the binding affinity of the receptor-binding domain to the angiotensin-converting enzyme 2 (ACE2) receptor. Four key concerns stemming from the emergence of the new variants are their effects on viral transmissibility, disease severity, reinfection rates (i.e., escape from natural immunity), and vaccine effectiveness (i.e., escape from vaccine-induced immunity).

The sa-RNA COVID VLP vaccines described herein can be adapted to encode any variant of the SARS-CoV-2 spike protein (based on reference protein found at Genbank: NC_045512), or any antigenic protein desired to be utilized as an “immune targeting” agent to stimulate host anti SARS-CoV-2 antibody production. Another advantage of the present sa-RNA VLPs includes the large size of the replicon for delivery of one or more immunogenic antigens. Further provided are fusion or chimeric antigens. For example, the replicon size can be anywhere from about 10,000-20,000 bp, with the payload being encoded by a polynucleotide of about 3-3.5 kb of nucleic acid. Additionally, a surprising observation is that an exemplary sa-RNA VLP, CB-106 is capable of inducing a cellular and/or humoral immune response to Severe Acute Respiratory Syndrome (SARS-CoV), and SARS-CoV-2, including inducing immune response against the South African, UK and Brazilian variants of SARS-CoV-2) in test subjects. Thus, illustrating the flexibility and widely applicable platform of utilizing the saRNA VLP's as described herein for generating protective immune responses, including against SARS variants.

A listing of emerging variants can be found online at: covariants.org. and are summarized in the table below.

TABLE 2

| Variant | First detected | Key mutations |
|---|---|---|
| B.1.526 | New York/November 2020 | S: L5F |
| 20C/S: 484K | | S: T95I |
| | | S: D253G |
| | | S: E484K |
| | | S: A701V |
| | | ORF1b: Q1011H |
| | | ORF3a: P42L |
| | | ORF1a: S3675- |
| | | ORF1a: G3676- |
| | | ORF1a: F3677- |
| | | N: P199L |
| | | N: M234I |
| | | ORF8: T11I |
| | | C241T |
| | | C3037T |
| | | A20262G |
| | | A28271- |
| B.1.525 | New York/December 2020 | S: A67V |
| 20A/S: 484K | | S: H69- |
| | | S: V70- |
| | | S: Y144- |
| | | S: E484K |
| | | S: Q677H |
| | | S: F888L |
| | | N: S2- |
| | | N: D3Y |
| | | N: A12G |
| | | ORF1a: S3675- |
| | | ORF1a: G3676- |
| | | ORF1a: F3677- |
| | | C241T |
| | | C1498T |
| | | A1807G |
| | | G2659A |
| | | C3037T |
| | | T8593C |
| | | C9593T |
| | | C18171T |
| | | A20724G |
| | | C24748T |
| | | A28699G |
| | | G29543T |
| P.2 | Brazil/April 2020 | Spike: E484K, |
| | | D614G, V1176F |
| | | ORF1a: L3468V, |
| | | L3930F |
| | | ORF1b: P314L |
| | | N: A119S, R203K, |
| | | G204R, M234I |
| | | 5′UTR: R81C |
| B.1.1.7 | United Kingdom | Δ69/70 |
| 201/501Y.V1 | | Δ144Y |
| | | (E484K*) |
| | | (S494P*) |
| | | N501Y |
| | | A570D |
| | | D614G |
| | | P681H |
| | | C241T |
| | | C913T |
| | | C3037T |
| | | C5986T |
| | | C14676T |
| | | C15279T |
| | | T16176C |
| | | ORF1a: S3675- |
| | | ORF1a: G3676- |
| | | ORF1a: F3677- |
| | | N: R203K |
| | | N: G204R |
| | | ORF8: Q27* |
| P.1 | Japan/Brazil | S: L18F |
| 20J/501Y.V3 | | S: K417T |
| | | S: E484K |
| | | S: N501Y |
| | | S: H655Y |
| | | ORF1a: S3675- |
| | | ORF1a: G3676- |
| | | ORF1a: F3677- |
| | | N: P80R |
| | | N: R203K |
| | | N: G204R |
| | | D614G |
| | | C241T |
| | | T733C |
| | | C2749T |
| | | C3037T |
| | | A6319G |
| | | A6613G |
| | | C12778T |
| | | C13860T |
| | | A28877T |
| | | G28878C |
| B.1.351 | South Africa | D80A |
| 20H/501Y.V2 | | S: D215G |
| | | S: L241- |
| | | S: L242- |
| | | S: A243- |
| | | S: K417N |
| | | S: E484K |
| | | S: N501Y |
| | | S: A701V |
| | | ORF1a: S3675- |
| | | ORF1a: G3676- |
| | | ORF1a: F3677- |
| | | N: T205I |
| | | E: P71L |
| | | D614G |
| | | G174T |
| | | C241T |
| | | C3037T |
| | | C28253T |
| B.1.427 | US-California | L452R |
| | | D614G |
| B.1.429 | US-California | S: S13I |
| 20C/S: 452R | | S: W152C |
| | | S: L452R |
| | | ORF1a: I4205V |
| | | ORF1b: D1183Y |
| | | D614G |
| | | C2395T |
| | | T2597C |
| | | T24349C |
| | | G27890T |
| | | A28272T |
| 20E (EU1) | Spain-EU | S: A222V |
| | | ORF10: V30L |
| | | N: A220V |
| | | T445C |
| | | C6286T |
| | | C26801G |
| 20A EU2 | EU | S: S477N |
| | | N: M234I |
| | | N: A376T |
| | | ORF1b: A176S |
| | | ORF1b: V767L |
| | | ORF1b: K1141R |
| | | ORF1b: E1184D |
| | | C4543T |
| | | G5629T |
| | | C11497T |
| | | T26876C |
| 20A/S: 439K | Ireland/Scotland | S: S439K |
| | | ORF1a: I2501T |
| | | C8047T |
| S: 677H.Robin1 | Wisconsin- US | S: Q677H |
| | | N: P67S |
| | | N: P199L |
| | | N: D377Y |
| | | ORF1a: T265I |
| | | ORF1a: L3352F |
| | | ORF1b: N1653D |
| | | ORF1b: R2613C |
| | | C241T |
| | | G1942T |
| | | C3037T |
| | | A9085G |
| | | C14805T |
| S: 677P.Pelican | US | S: Q677P |
| | | N: P67S |
| | | N: P199L |
| | | ORF1a: T265I |
| | | ORF1a: L3352F |
| | | ORF1b: N1653D |
| | | ORF1b: R2613C |
| | | C241T |
| | | C3037T |
| | | C21811A |
| | | T29194C |
| | | T29377A |
| 20A/S: 98F | Belgium and the Netherlands | S: S98F |
| | | N: P199L |
| | | ORF3a: Q38R |
| | | ORF3a: G172R |
| | | ORF3a: V202L |
| | | C28651T |
| 20C/S: 80Y | France/EU | S: D80Y |
| | | N: S186Y |
| | | N: D377Y |
| | | ORF1a: T945I |
| | | ORF1a: T1567I |
| | | ORF1a: Q3346K |
| | | ORF1a: V3475F |
| | | ORF1a: M3862I |
| | | ORF1b: P255T |
| | | ORF7a: R80I |
| | | G4960T |
| | | C6070T |
| | | C7303T |
| | | C7564T |
| | | C10279T |
| | | C10525T |
| | | C10582T |
| | | C27804T |
| 20B/S: 626S | Norway | S: 626S |
| 20B/S: 1122L | Sweden | S: V1122L |

### 4.12. Immunogenic and/or Antigenic Compositions and Vaccines and Administration

In one aspect, the disclosure provides a recombinant VLP particle that is capable of directing expression of a heterologous or foreign protein (e.g., a coronavirus protein) in the host or patient/subject as VLP vaccine.

In certain embodiments, an immunogenic and/or antigenic composition or vaccine is formulated such that the VLP vaccine, in which the RNA in the VLP replicon directs the production of at least one immunogenic antigen of an infectious agent in a target/host cell so as to elicit an immune (humoral and/or cell mediated) response in the target/host that is prophylactic or therapeutic. In an embodiment wherein the sa-RNA VLP delivers an RNA encoding at least one immunogenic antigen of a pathogen (e.g., SARS-Cov-2), administration of the sa-RNA VLP vaccine is carried out to prevent or treat an infection by the pathogen and/or the resultant infectious disorder and/or other undesirable correlates of infection.

In certain aspects, provided herein are compositions (e.g., pharmaceutical compositions, immunogenic compositions, VLP vaccines) comprising the recombinant VLP particles described herein and a carrier and/or excipient.

Administration of the recombinant VLP particles described herein can be used as a method of immunostimulation, to boost the host's immune system, enhancing cell-mediated and/or humoral immunity, and facilitating the clearance of infectious agents or symptoms of a disease or disorder in a subject infected with SARS-CoV-2 (e.g., having COVID-19). The present disclosure thus provides a method of immunizing an animal or treating or preventing various diseases or disorders in an animal, comprising administering to the animal an effective immunizing dose of a VLP vaccine of the present disclosure.

In certain aspects, the disclosure provides a method of treating or preventing a disease or disorder in a subject comprising administering to the subject an effective amount of the recombinant VLP particles described herein to induce an immune response (e.g., a protective immune response) against a foreign protein. In certain embodiments, the foreign protein is a coronavirus full-length spike protein, or the ectodomain, or a fragment or a derivative thereof. In a specific embodiment, the full-length spike protein, or the spike ectodomain is derived from SARS-CoV-2. In certain embodiments, the sa-RNA VLP is capable of eliciting an immune response to one or more variants of SARS-CoV-2 of in a subject. In additional embodiments, the RNA encoding at least one immunogenic antigen of a pathogen is modified to encode a variant spike protein or spike ectodomain to produce an sa-RNA VLP that is broadly protective against more than one variant of SARS-CoV-2.

In certain embodiments, the disclosure provides a method for the treatment or prevention of a disease or disorder in a subject infected with SARS-CoV-2. In certain embodiments, the disease or disorder is COVID-19.

In certain aspects, the disclosure provides a method of treating or preventing a disease or disorder in a subject comprising administering to the subject an effective amount of the recombinant VLP particles described herein to induce the formation of neutralizing antibodies against a foreign protein. In certain embodiments, the foreign protein is a coronavirus S glycoprotein, or a fragment or a derivative thereof. In a specific embodiment, the S glycoprotein is derived from SARS-CoV-2. In certain embodiments, the disclosure provides a method for the treatment or prevention of a disease or disorder in a subject infected with SARS-CoV-2. In certain embodiments, the disease or disorder is COVID-19.

In certain embodiments directed to vaccines, the recombinant VLP particles described herein are administered prophylactically, to prevent/protect against a SARS-CoV-2 infection and/or infectious disease (e.g., having COVID-19).

Many methods may be used to introduce the immunogenic and/or antigenic compositions and vaccines described herein, such as, but not limited to oral, intradermal, intramuscular, intraperitoneal, intravenous, infusions, subcutaneous, intranasal routes, and via scarification (scratching through the top layers of skin, e.g., using a bifurcated needle).

In certain embodiments, the delivery route is intramuscular (IM). The muscles have a plentiful supply of blood, which helps ensure that the body absorbs the vaccine quickly. The tissue in the muscles can also hold more medication than fatty tissue. In certain embodiments, intramuscular injection is followed by electroporation.

The subject to which the VLP vaccine is administered can include humans, non-human primates, veterinary animals (e.g., cats, dogs, cows, horses, sheep, pigs, goats, hamsters, etc.), and experimental animal models of diseases (e.g., mice, rats, ferrets, monkeys, etc.). In a preferred embodiment, the subject is a human.

In certain embodiments, the VLP vaccines described herein comprise an effective amount of the recombinant VLP particles described herein and a pharmaceutically acceptable carrier or excipient. Pharmaceutically acceptable carriers are well known in the art and include, but are not limited to, saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, etc. The carrier is preferably sterile. The formulation should suit the mode of administration, which is readily determined by one of skill in the art.

In certain embodiments, the VLP vaccine can also comprise minor amounts of wetting or emulsifying agents, or pH buffering agents. The immunogenic and/or antigenic composition or vaccine can be a liquid solution, suspension, emulsion, tablet, pill, capsule, sustained release formulation, or powder. Oral formulations can include one or more standard carriers such as pharmaceutical grades of mannitol, lactose, starch, gelatin, magnesium stearate, sodium saccharine, cellulose, magnesium carbonate, methylcellulose (e.g., 4000 cP, 25 cP, METHOCEL™ E3, E5, E6, E15, E50, E4M, E10M, F4, F5, F4M, K3, K100, K4M, K15M, K100M, K4M CR, K15M CR, K100M CR, E4M CR, E10M CR, K4M Premium, K15M Premium, K100M Premium, E4M Premium, E10M Premium, K4M Premium CR, K15M Premium CR, K100M Premium CR, E4M Premium CR, E10M Premium CR, and K100 Premium LV), monosodium glutamate, human serum albumin, fetal bovine serum, trehalose, alginate (e.g., BioReagent), guar gum, MUCOLOX™, etc. In certain embodiments, the formulation has an appropriate viscosity to maintain stability of the virus particles. In certain embodiments, the formulation has an appropriate carrier to allow the viral particles to maintain contact with mucosal membranes for an appropriate amount of time for them to be taken up.

The ingredients are supplied either separately or mixed together in unit dosage form, for example, as a dry lyophilized powder or water free concentrate in a hermetically sealed container such as an ampoule or sachette indicating the quantity of active agent. In certain embodiments where in the immunogenic and/or antigenic composition or vaccine is administered by injection, an ampoule of sterile diluent can be provided so that the ingredients may be mixed prior to administration.

In certain embodiments, lyophilized recombinant VLP particles described herein are provided in a first container and a second container comprises diluent (e.g., an aqueous solution of 50% glycerin, 0.25% phenol, and an antiseptic (e.g., 0.005% brilliant green)).

The precise dose of virus, or subunit vaccine, to be employed in the VLP vaccine will also depend on the route of administration, and the nature of the patient, and should be decided according to the judgment of the practitioner and each patient's circumstances according to standard clinical techniques. The VLP vaccine is administered in an amount sufficient to produce an immune response to the foreign protein in the host to which the recombinant VLP particle is administered.

In certain embodiments, the immunogenically and/or antigenically effective amount can comprise a dosage of about 100 pg to 1000 ng of RNA equivalent of the VLP which is effective for generating antibodies to the antigen encoded by the VLP replicon. Additional dosages include 10-100 ng of RNA equivalent of the VLP, or more preferably a dose of at least 100 ng, 150 ng, 200 ng, 225 ng, 250 ng, 275 ng, or at least 500 ng, which provides an acceptable efficacy of protection against the desired infection. In certain embodiments, effective doses of the immunogenic and/or antigenic composition or vaccine described herein may also be extrapolated from dose-response curves derived from animal model test systems. Such effective dose is considered a very low dose, and the ability of such a low dose to elicit a protective immune response in a patient/subject is unexpected.

In certain embodiments, a boosting dose is used. In certain embodiments, the boosting dose can be any SARS-CoV-2 vaccine. In certain embodiments, the boosting dose comprises any of the recombinant VLP particle vaccines described herein. In certain embodiments, the boosting dose comprises the foreign protein or peptide in purified form, or a nucleic acid encoding the foreign protein or peptide, rather than using a recombinant VLP particle described herein. In certain embodiments, the boosting dose comprises the same SARS-COV-2 vaccine as the SARS-COV-2 vaccine it is boosting (i.e. the vaccines are homologous). In certain embodiments, the boosting dose comprises a SARS-COV-2 vaccine that is different than the SARS-COV-2 vaccine it is boosting (i.e., the vaccines are heterologous).

In certain embodiments, the boosting dose comprises any of the recombinant VLP particle vaccines described herein. In certain embodiments, the boosting dose is used to boost any of the recombinant VLP particle vaccines described herein. In certain embodiments, the boosting dose is used to boost a SARS-CoV-2 vaccine other than the recombinant VLP particle vaccines described herein.

Many methods may be used to introduce the boosting dose, such as, but not limited to oral, intradermal, intramuscular, intraperitoneal, intravenous, infusions, subcutaneous, intranasal routes, and via scarification. In certain embodiments, the delivery route is oral or mucosal (whether oral or intranasal). In certain embodiments, oral delivery may comprise application on a solid physiologically acceptable base, or in a physiologically acceptable dispersion. In certain embodiments, oral delivery may comprise administering the dose in a fluid form. In certain embodiments, the delivery route is intramuscular.

In certain embodiments, the boosting dose is administered after a single dose of the SARS-CoV-2 vaccine. In certain embodiments, boosting dose is administered after repeated doses of the SARS-CoV-2 vaccine (e.g., 2, 3, 4, or 5 doses). The period of time between SARS-COV-2 vaccine administration and the boosting dose can be 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, or longer. If more than one boost is performed, the subsequent boost can be administered 1 week, 2 weeks, 3 weeks, 4 weeks, 5 weeks, 6 weeks, 7 weeks, 8 weeks, 3 months, 4 months, 5 months, 6 months, or longer after the preceding boost. For example, the interval between any two boosts can be 4 weeks, 8 weeks, or 12 weeks. For example, the SARS-COV-2 vaccine may be administered twice (e.g., via injection) before the boosting dose is administered (e.g., orally) and the boost is repeated every 3 months.

In certain embodiments, the priming dose comprises any of the recombinant VSV particle vaccines described herein. In certain embodiments, the priming dose is used to prime any of the recombinant VLP vaccines described herein. In certain embodiments, the priming dose is used to prime a SARS-CoV-2 vaccine other than any of the recombinant sa-RNA VLP vaccine described herein. In certain embodiments, the priming dose comprises the same SARS-COV-2 vaccine as the SARS-COV-2 vaccine it is priming. In certain embodiments, the priming dose comprises a SARS-COV-2 vaccine that is different than the SARS-COV-2 vaccine it is priming.

An advantage of the present sa-RNA VLP system is that it is not limiting for additional treatments—be they AAV based, or other RNA based vaccines. The sa-RNA VLP backbone does not produce substantial cross-reacting antibodies, that would inhibit or affect administration of vaccines that use a different platform or backbone. Additionally, there are no limitations on future vaccines or treatment using the sa-RNA VLP system or platform.

Many methods may be used to introduce the priming dose, such as, but not limited to oral, intradermal, intramuscular, intraperitoneal, intravenous, infusions, subcutaneous, intranasal routes, and via scarification. In certain embodiments, the delivery route is oral or mucosal (whether oral or intranasal). In certain embodiments, oral delivery may comprise application on a solid physiologically acceptable base, or in a physiologically acceptable dispersion. In certain embodiments, oral delivery may comprise administering the dose in a fluid form. In certain embodiments, the priming dose is administered via intramuscular injection.

The period of time between the VLP priming dose and VLP vaccine administration can be 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, or longer. For example, the interval between the VLP priming dose and the vaccine can be 4 weeks, 8 weeks, or 12 weeks. For example, the VLP priming dose may be administered (e.g., via injection) before the VLP vaccine is administered. In additional embodiments, the interval between any two boosts can be 4 weeks, 8 weeks, or 12 weeks. For example, sa-RNA VLP vaccine may be administered twice (e.g., via injection) before the boosting dose is administered (e.g., orally) and the boost is repeated every 3 months.

In certain embodiments, a prime dose and boost dose are used. In certain embodiments, the priming dose can be any of the recombinant VLP particle vaccines described herein, and the boosting dose can be any of the recombinant VLP particle vaccines described herein. In certain embodiments the prime and boost at the same vaccine, and in alternative embodiments the prime and boost are heterologous vaccines.

### 4.13. Stability of sa-RNA VLP Vaccine Compositions

In certain aspects, the disclosure provides VLP compositions that are stable at from about 4°−10° C. In certain embodiments, the VLP compositions are stable at from about 4°-10° C. to at least about one week, at least about ten days, at least about two weeks, at least about three weeks, at least about four weeks, at least about five weeks, at least about six weeks, at least about seven weeks, at least about eight weeks, at least about nine weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, at least about 12 months, or at least about 2 years.

In another aspect, the disclosure provides VLP compositions that allow at least 3 freeze/thaw cycles of the VLPs while maintaining activity. In certain embodiments, the vaccine formulation allows for at least 3 freeze/thaw cycles of the virus particles while maintaining at least about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% activity. In certain embodiments, the vaccine formulations allow at least 3 freeze/thaw cycles of the virus particles while maintaining at least about 30% activity.

### 4.14. Antibodies Subsequently Generated by Subjects/Patients Administered the sa-RNA VLP Vaccine Compositions

In one aspect, the disclosure provides a method for generating antibodies against the at least one immunogenic antigen of an infectious agent using the recombinant VLP particles described herein. The generated antibodies may be isolated by standard techniques known in the art (e.g., immunoaffinity chromatography, centrifugation, precipitation, etc.).

Antibodies generated against the at least one immunogenic antigen of an infectious agent by immunization with the recombinant VLP vaccines described herein also have potential uses in diagnostic immunoassays and passive immunotherapy.

Assays in which the antibodies generated by hosts inoculated with the recombinant VLP vaccine described herein can include, but are not limited to, competitive and noncompetitive assay systems using techniques such as radioimmunoassays, ELISA (enzyme-linked immunosorbent assays), “sandwich” immunoassays, precipitin reactions, gel diffusion precipitin reactions, immunodiffusion assays, agglutination assays, complement-fixation assays, immunoradiometric assays, fluorescent immunoassays, protein A immunoassays and immunoelectrophoresis assays, etc.

### 4.15. Kits

Additionally, certain components or embodiments of the compositions can be provided in a kit. For example, the sa-RNA VLP vaccine composition, as well as the related buffers or other components related to administration can be provided in separate containers and packaged as a kit, alone or along with separate containers of any of the other agents from any pre-conditioning or post-conditioning steps, and optional instructions for use. In some embodiments, the kit may comprise ampoules, disposable syringes, capsules, vials, tubes, or the like. In some embodiments, the kit may comprise a single dose container or multiple dose containers comprising the embodiments herein. In some embodiments, each dose container may contain one or more unit doses. In some embodiments, the kit may include an applicator.

In some embodiments, the kits include all components needed for the various stages of treatment. In some embodiments, the compositions may have preservatives or be preservative-free (for example, in a single-use container). In some embodiments, the kit may comprise materials for intramuscular administration. In some embodiments, the kit may comprise buffers, diluents, or emergency allergic reaction treatments, such as epinephrine or Benadryl in a separate container.

### 5. Examples

### Example 1

### 5.1. In-Vivo Study in Mice to Determine Neutralizing Antibody Generation Against SARS-COV-2 Using VLP-saRNA

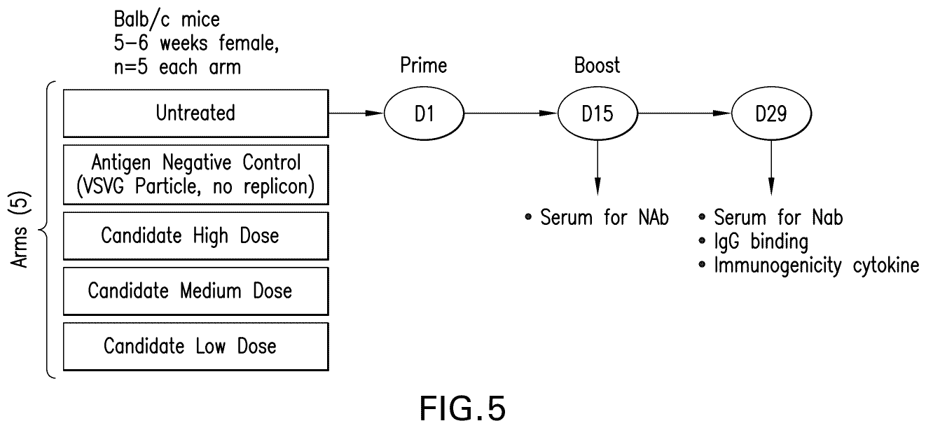

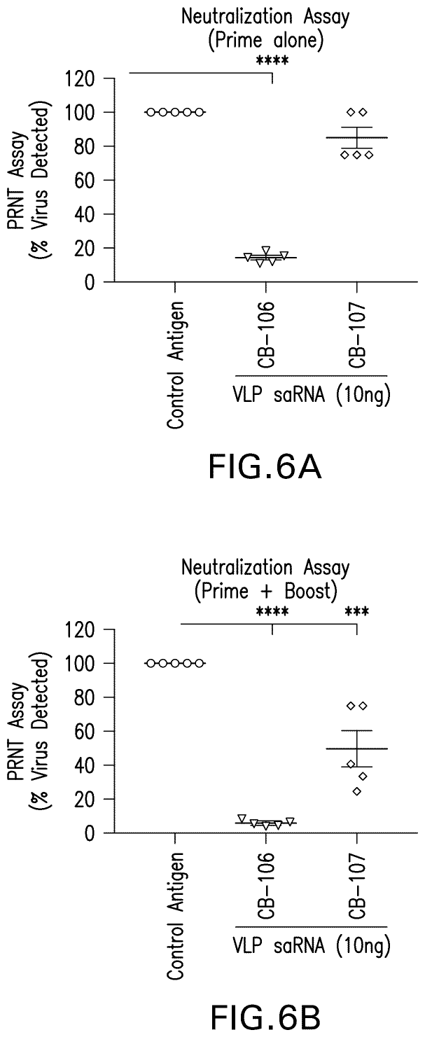

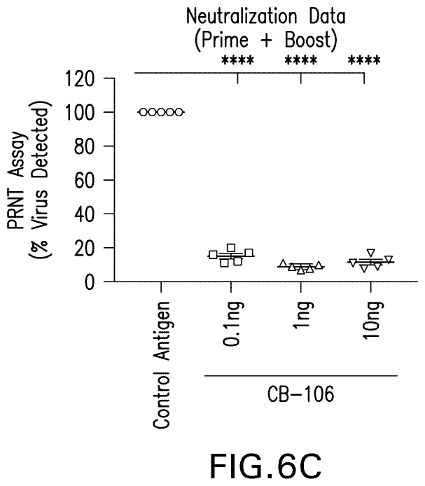

A prime and boost study was designed in 7-week-old female Balb-c mice to test the ability of the vaccines (VLP-saRNA candidates) to generate neutralizing antibodies against SARS-COV-2 (FIG. 5). The candidate designs are shown in FIG. 2, and various sequence options in FIGS. 3A-3G. Mice (n=5 or 7) were injected intramuscularly with 50 μl of either vaccine at Day 1 (Prime) and Day 14 (Boost) in a 4-week study. Serum samples were collected pre-boost and at the end of study to determine neutralizing antibodies and induction of immunoglobulins. Data obtained were compared to a negative antigen control cohort. Dose was calculated based on infectivity assays for VLP-saRNA. These data are shown in FIGS. 6A-6C. Candidate construct 1 (CB-106) contains the full-length codon optimized polynucleotide encoding full length spike protein which is encapsidated in the replicon as RNA. Candidate construct 2 (CB-107) contains a codon optimized polynucleotide (SEQ ID NO:9) encoding the spike protein ectodomain (SEQ ID NO:10) encoded in the replicon. Candidate 1 has provided the best observed data and is being termed CB-106.

### 5.2. Plaque Reduction Neutralization Assay

African green monkey kidney (Vero E6) cells were grown to confluency in 12-well tissue-culture plates. Titration of each serum were performed in triplicate. The serum samples for Plaque Reduction Virus Neutralization (PRNT) assay were heat-inactivated at 56° C. for 30 minutes and diluted 1:10 or more to determine live virus neutralizing capabilities. 200 plaque forming units (PFUs) of SARS-CoV-2 was added to the diluted (1:10) serum and incubated at 37° C. for 1 hour and then plated onto confluent Vero E6 cells. The plates were overlaid with semi-solid media, incubated for 48 hours and then stained with neutral red and plaques counted. Data is reported as % PFUs or Virus detected.

### 5.3. Antigen Specific IgG Elisa

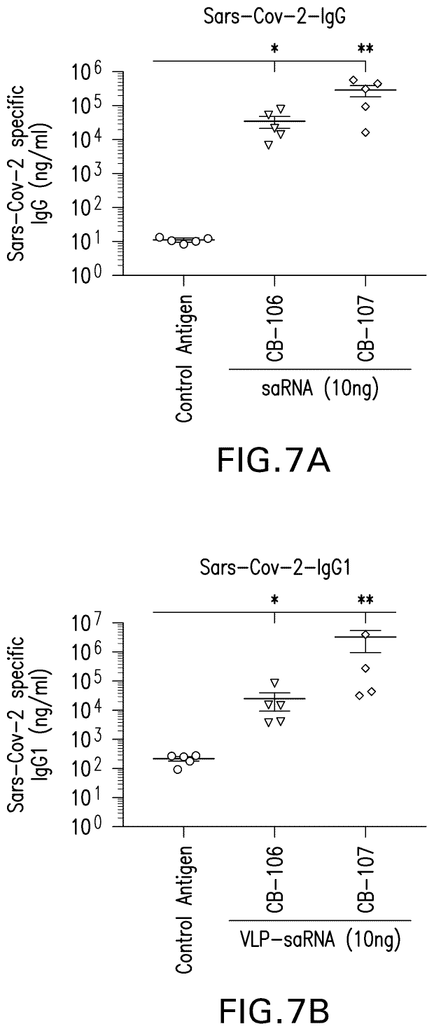

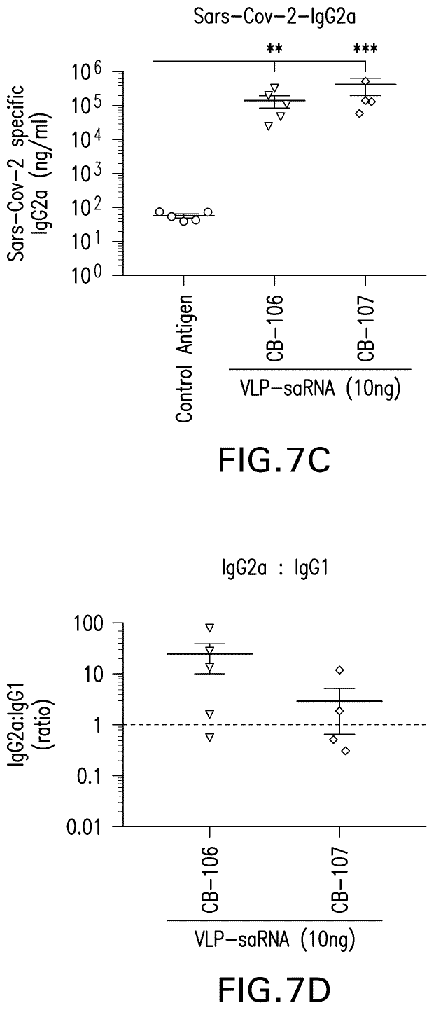

The antigen-specific IgG, IgG1 and IgG2a titers in mouse sera were assessed by a semi-quantitative ELISA. Protocol used was adapted from McKay P F et.al, Nature Communication, 2020 (https://doi.org/10.1038/s41467-020-17409-9). Briefly, high binding ELISA plates were coated with 100 μL per well of 1 μg/mL recombinant SARS-CoV-2 protein in PBS. For the standard IgG/IgG1/IgG2a, 3 columns on each plate were coated with 1:1000 dilution each of goat anti-mouse Kappa and Lambda light chains. After overnight incubation at 4° C., the plates were washed 4 times with PBS-Tween 20 0.05% (v/v) and blocked for 1 h at 37° C. with 200 μL per well blocking buffer (1% BSA (w/v) in PBS-Tween-20 0.05% (v/v)). The plates were then washed and the diluted samples or a 5-fold dilution series of the standard IgG (or IgG1 or IgG2) added using 50 μL per well volume. Plates were incubated for 1 h at 37° C., then washed and secondary antibody added at 1:2000 dilution in blocking buffer (100 μL per well) using either anti-mouse IgG-HRP, anti-mouse IgG1-HRP or anti-mouse IgG2a-RP. After incubation and washes, plates were developed using 50 μL per well TMB (3,3′, 5,5′-tetramethylbenzidine) substrate and the reaction stopped after 5 min with 50 μL per well stop solution. The absorbance was read on a Spectrophotometer at 450 nm. These data are shown in FIGS. 7A-7D, which illustrate the Th1/Th2 skew in response to SARS-CoV-2 VLP-saRNA vaccine. IgG1 (FIG. 7B) and IgG2a (FIG. 7C) responses in mice vaccinated with VLP-saRNA. N=5 animals. The mice demonstrated a Th1 bias (FIG. 7D) Data shown as Mean±SD. Significance compared to antigen control was determined by performing a one-way ANOVA; **p<0.001, ***p<0.0001.

### 5.4. Particle Manufacturing and Concentration

### 5.4.1. SARS-CoV-2 VLP-saRNA Particle

Three plasmids individually coding for RSV-GAG, VSV-G and VEEV replicon with Spike gene as GOI in the ratio of 1:1:1 or others were electroporated into a production cell line (BHK-21, BHK/AC9, HEK293, VERO, A549 among others) and the cells incubated for 48-96 hours. In addition to electroporation, PEI (polyethylenimine) based transfection was also used. In this case, different ratios of PEI:DNA was used including 1:1, 1:2, 1:3. After incubation, the cells and media was collected and spun down at 1000 xg for 10 min. The purified supernatant was then subjected to ultracentrifugation (134000 xg for 4 hrs) in 20% glycerol. The pellet formed was dissolved in 1×PBS and stored at −80° C. In some instances, the supernatant was subjected to anion exchange resins, purified using HPLC and TFF prior to concentration and storage at −80° C.

### 5.5. Temperature Stability Experiments

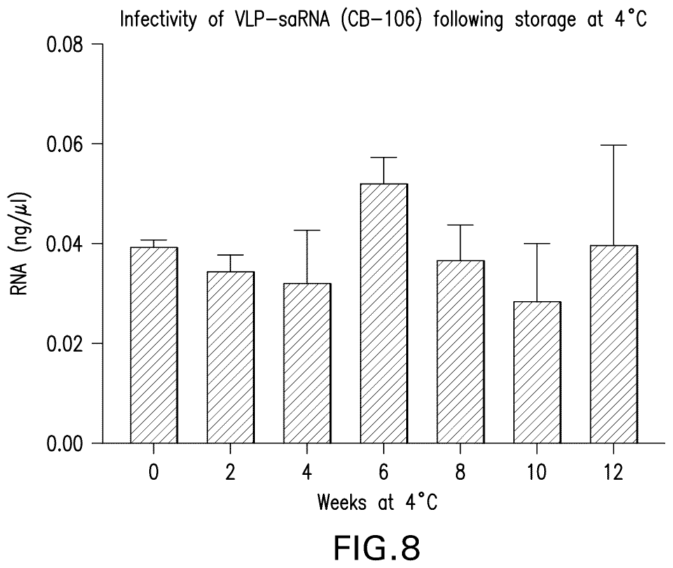

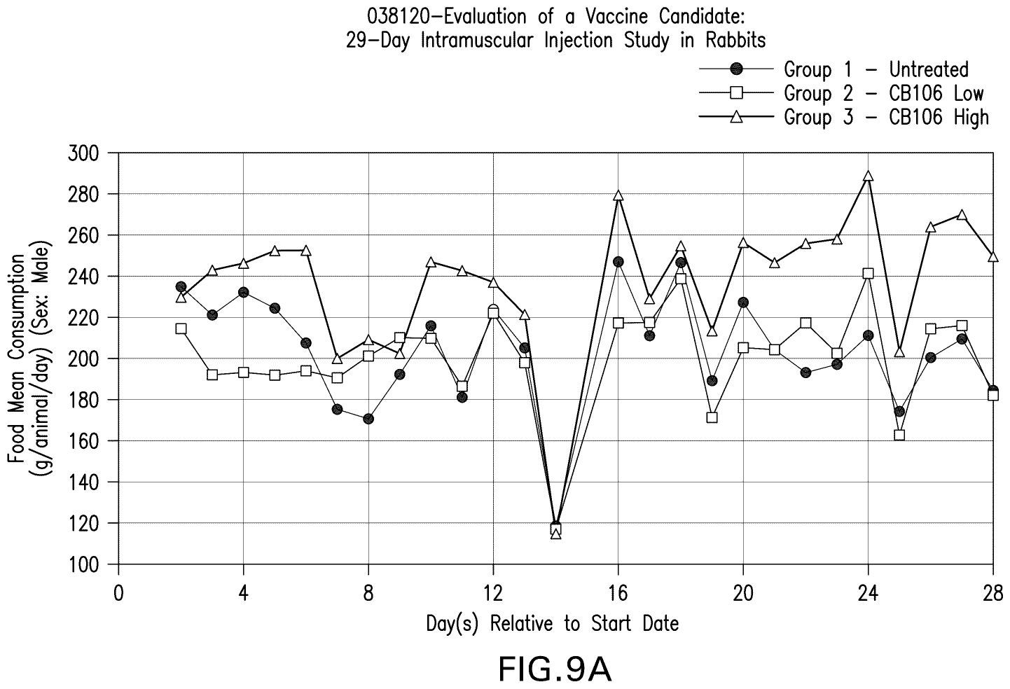

The purified and concentrated VLP-saRNA vaccines were stored at 4° C. Aliquots were collected at different times and measured for stable gene expression (VLP-saRNA). To determine the stability of the VLP-saRNA, an aliquot was diluted 1:100 and added to BHK-21 cells. Twenty-four hours later, cells were collected, RNA isolated and quantified using quantitative RT-PCR. Stability of VLP-protein was determined by western blot analysis or SARS-COV-2 Spike specific ELISA. Stability has been demonstrated at from about 2° C.-8° C. for at least one-six months; and at about −80° C. for at least about 2 years. FIG. 8 shows the RNA stability measured by infectivity assay for up to twelve weeks.

### 5.6. VLP-saRNA Safety/Toxicity Studies in Rabbits

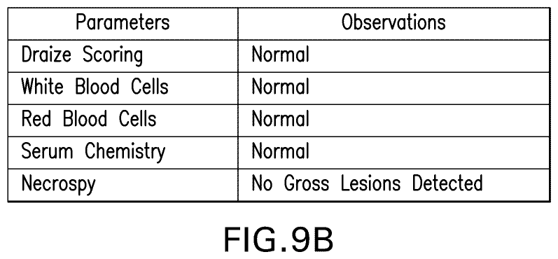

Two doses (VLP-saRNA-150 ng and 15 ng) of each of the vaccine candidates were each administered, on Days 1 and 15 via intramuscular injection (0.5 mL) in the right hindlimb, to different groups of two male and female New Zealand White (NZW) rabbits. Dose selected was the human equivalent dose calculated from the mice studies. The control NZW rabbits (2/sex) were untreated. All rabbits were terminated on Day 29. Animals were monitored for clinical observations twice a day, predose detailed clinical observations, dermal observations (Draize scores) at the injection site prior to and for 3 days following each dose, weekly body weights, daily quantitatively assessment of food consumption, body temperature prior to each dose and daily thereafter until the temperature of all animals was within the normal range (38-39.9° C.), and clinical pathology (hematology and serum chemistry parameters) on Days 15 and 29. Postmortem assessment included gross necropsy, measurement of selected organ weights (heart, kidneys, liver, lung, spleen, and thymus), and microscopic evaluation of a selected tissues (brain, gallbladder, heart, injection site, kidneys, liver, lungs, ovaries, spleen, testes, and thymus). Samples for immunogenicity tests (Sars-Cov-2 neutralizing antibody and Sars-Cov-2 specific IgG) were collected from all animals on Day 1 (predose), Day 15 (predose), and Day 29. These data are shown in FIGS. 9A-9B, where there was no impact on body weight gain, food consumption (FIG. 9A) and other parameters measured (FIG. 9B).

### Example 2

### 5.7. Clinical Phase-1 Design for SARS-CoV-2 saRNA Vaccine (Lead VLP CB-106

The Phase-1 study will evaluate the safety, reactogenicity and immunogenicity of a lead SARS-CoV-2 saRNA VLP vaccine named CB-106 in a dose-ranging study. Three dose levels of CB-106 will be given to healthy adults in two doses, 28 days apart, and three dose levels of CB-106 will be administered as single dose to healthy adults. Assessments will be conducted in a 7-Arm study, n=175 volunteer, approximately 25 volunteers enrolled in each arm, open-label, dose-ranging study involving men and women ages 18 and up who meet all eligibility criteria, defined below. Dose range will be evaluated in a Prime alone (single dose) and a Prime and Boost (2 dose) regimen. Doses will be selected on Human Dose Equivalent calculations based on concentrations in mice and is expected to range from 100 picograms (0.0001 microgram) to 100 micrograms of RNA. Each dose will be administered as 0.3 mL-0.5 mL vaccine via intra-muscular injection on Day 1 for the prime only cohort and Days 1 and 29 for the booster cohort. Subjects will be monitored up to 12 months post-vaccination (Day 394), including visits 1, 2, and 4 weeks after each vaccination, and three and six months after the second vaccination. Primary and Secondary outcomes measured up to Day 57 (28 days after the booster dose on Day 29) may be used to generate preliminary critical data package to secure Fast Track designation and approval to recruit to Phase-II volunteer study.

Experimental Arm One: CB-106 Dose Level 1: A single value within the range of 0.0001 microgram to twenty (20) micrograms of RNA will be tested.
Participants will receive 2 intramuscular (IM) injections of CB-106 at Dose Level 1 on Day 1 and Day 29.

Experimental Arm Two: CB-106 Dose Level 2: A single value within the range of twenty (20) micrograms to sixty (60) micrograms of RNA will be tested.
Participants will receive 2 IM injections of CB-106 at Dose Level 2 on Day 1 and Day 29.

Experimental Arm Three: CB-106 Dose Level 3: A single value within the range of sixty (60) micrograms to One hundred (100) micrograms of RNA will be tested.
Participants will receive 2 IM injections of CB-106 at Dose Level 3 on Day 1 and Day 29.

Experimental Arm Four: CB-106 Dose Level 1: A single value within the range of 0.0001 microgram to twenty (20) micrograms of RNA will be tested.
Participants will receive 1 IM injection of CB-106 at Dose Level 1 on Day 1 and 1 IM injection of study drug-matching placebo on Day 29.

Experimental Arm Five: CB-106 Dose Level 2: A single value within the range of twenty (20) micrograms to sixty (60) micrograms of RNA will be tested.
Participants will receive 1 IM injection of CB-106 at Dose Level 2 on Day 1 and 1 IM injection of study drug-matching placebo on Day 29.

Experimental Arm Six: CB-106 Dose Level 3: A single value within the range of sixty (60) micrograms to One hundred (100) micrograms of RNA will be tested.
Participants will receive 1 IM injection of CB-106 at Dose Level 3 on Day 1 and 1 IM injection of study drug-matching placebo on Day 29.

Experimental Arm Seven: Study-Drug Matched Placebo
Participants will receive 2 intramuscular (IM) injections of Study-Drug matched placebo on Day 1 and Day 29.

Primary Outcome measures will include:
1. Number of participants with solicited local and systemic reactogenicity adverse reactions (ARs.) This will be monitored up to Day 36 in the case of prime and boost study arms (7 days after the booster dose on Day 29) and up to Day 7 after the prime dose in case of Prime only arms.
2. Number of participants with unsolicited adverse events (AEs.) This will be monitored up to Day 57 in the case of prime and boost study arms (28 days after the booster dose on Day 29) and up to Day 28 after the prime dose in case of Prime only arms.
3. Number of participants with medically attended AEs (AAAEs), AE of Special Interest (AESIs) and Serious Adverse Events (SAEs). This will be monitored up to Day 394 after the second close in the case of prime and boost study arms.
4. It is expected that only Grade 1 or Grade 2 reactions will be observed following prime and/or boost administrations, and no serious adverse reactions, such as AAAEs, AESIs, or SAEs are expected in view of the current animal data (See, FIG. 9A-B).

Secondary Outcome measures will include:
1. Geometric Mean (GM) of SARS-CoV2 specific neutralizing antibodies (nAb) on Day 1, Day 29, Day 36, Day 57, Day 209 and Day 394.
2. GM of SARS-CoV-2 specific binding antibody (bAb) on Day 1, Day 29, Day 36, Day 57, Day 209 and Day 394.
3. Seroconversion as measured by an increase of SARS-CoV-2 specific nAb titer or bAb titer on Day 1, Day 7, Day 29, Day 36, Day 209 and Day 394.

Key Inclusion Criteria for each Subject/Patient:
Understands and agrees to comply with the study procedures and provides written informed consent.
According to the assessment of the investigator, is in good general health and can comply with study procedures.
Body mass index (BMI) of 18 kilograms/square meter (kg/m\hat{2}) to 35 kg/m\hat{2} (inclusive) at the Screening Visit (Day 0).
For female participants of childbearing potential: negative pregnancy test, adequate contraception or has abstained from all activities that could result in pregnancy for at least 28 days prior to the first injection, agreement to continue adequate contraception or abstinence through 3 months following the second injection, and not currently breastfeeding.

Key Exclusion Criteria for each Subject/Patient:
Known history of SARS-CoV-2 infection or known exposure to someone with SARS-CoV-2 infection or COVID-19 in the past 30 days.
Positive serology results for SARS-CoV-2 at the Screening Visit. A negative serological test for SARS-CoV-2, performed on a blood sample obtained at the Screening Visit, is required before a participant can be dosed.
Travel outside of the region of participation in the 28 days prior to the Screening Visit (Day 0).
Prior administration of an investigational, authorized, or licensed CoV (for example, SARS-CoV-2, SARS-CoV, or Middle East Respiratory Syndrome [MERS]-CoV) vaccine, based on medical history interview.
Current treatment with investigational agents for prophylaxis against COVID-19.
Recent (within the last 12 months) use of a dermal filler.
Has a medical, psychiatric, or occupational condition that may pose additional risk as a result of participation or that could interfere with safety assessments or interpretation of results according to the investigator's judgment.
Has received systemic immunosuppressants or immune-modifying drugs for >14 days in total within 6 months prior to Screening (for corticosteroids, 10 milligrams (mg)/day of prednisone equivalent) or is anticipating the need for immunosuppressive treatment at any time during participation in the study.
Has received or plans to receive any licensed vaccine 28 days prior to the first injection (Day 1) or plans to receive a licensed vaccine within 28 days before or after any study injection, with the exception of licensed influenza vaccines, which may be received more than 14 days before the first study injection or more than 14 days after the second study injection.
Receipt of systemic immunoglobulins or blood products within 3 months prior to the Screening Visit (Day 0) or plans for receipt during the study.
Current use of any inhaled substance (for example, tobacco or cannabis smoke, nicotine vapors).
History of chronic smoking (1 cigarette a day) within 1 year of the Screening Visit.
Resides in a nursing home.
Has donated 450 milliliters (mL) of blood products within 28 days prior to the Screening Visit or plans to donate blood products during the study.
Participated in an interventional clinical study within 28 days prior to the Screening Visit based on the medical history interview or plans to do so while participating in this study.

SEQUENCE DESCRIPTIONS
SEQ ID NO: 1
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVENATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNOVAVLYQDVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLFNKVTLADAGFIKQYGDCLGDIAARDLICAQKF
NGLTVLPPLLTDEMIAQYTSALLAGTITSGWTFGAGAALQIPFAM
QMAYRFNGIGVTQNVLYENQKLIANQFNSAIGKIQDSLSSTASAL
GKLQDVVNQNAQALNTLVKQLSSNFGAISSVLNDILSRLDKVEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFDEDD
SEPVLKGVKLHYT
COVID-19 Spike Glycoprotein (S-protein)
Amino acid sequence
S1 domain underlined
SEQ ID NO: 2
atgtttgtgttcctggtgcttctgccacttgtttcctcacaatgc
gtcaatctcacaacaagaacgcagcttccgcctgcatatacaaat
agtttcaccagaggcgtttattatccagataaggtctttcgaagc
tctgtcctccattctacgcaagatctgttcttgccattcttttca
aacgtgacgtggtttcacgctattcacgtttctggcactaatggc
acgaagcgctttgacaaccccgtccttccgttcaacgatggcgtc
tatttcgcgagtacagagaagtcaaatataatccgcggctggata
tttggcacaaccctcgatagtaaaacacaatcccttctgatcgta
aataacgcaacgaatgtggtgattaaggtatgcgagtttcaattt
tgcaacgacccgttccttggcgtttattatcacaaaaataacaag
tcctggatggagtccgaattcagagtgtattcttctgccaacaat
tgcacctttgagtacgtgtcccagcccttcttgatggatctggaa
ggaaaacagggcaatttcaagaatttgagagagttcgtcttcaaa
aacatcgatggttattttaagatctatagcaagcacaccccgata
aacctcgtacgggaccttccccaggggtttagcgcactggagcct
cttgtagatctcccgattggcatcaatataaccaggtttcaaact
cttcttgcacttcaccgaagctatcttacgcccggcgatagctct
tccggttggacagccggcgccgcggcctactatgtgggatatctc
cagcctagaacctttctgcttaagtacaatgaaaacggcactatt
acagacgccgtagattgcgctctggatcctcttagcgaaactaag
tgtactctgaaatcttttacggtagagaagggaatttatcaaaca
agtaatttccgggtacaacccacagagagcatcgtccggtttccg
aacattacgaatctttgcccgtttggcgaagtcttcaatgcaaca
cgcttcgcttccgtttatgcgtggaatcgcaagaggatctctaac
tgcgttgctgactacagtgttctttataacagcgctagcttttct
acattcaaatgttacggggtaagccccaccaagctcaacgacttg
tgttttaccaatgtttatgcggattctttcgtgattcgcggtgat
gaagttaggcagattgcgccaggacagactgggaagattgccgac
tataactataagctccctgacgacttcactgggtgtgtcattgct
tggaacagcaacaatctcgacagcaaagttggaggcaactataat
tatctctaccggctttttcgcaagtctaacctcaagcccttcgaa
agagacatcagtactgagatctatcaagcaggaagcacgccatgc
aacggtgtcgagggcttcaactgttattttcctcttcaatcctac
ggctttcaaccgacaaacggcgtcggatatcagccttatcgagtc
gttgtactttctttcgaacttctgcacgcaccagctactgtctgc
gggcctaaaaaatctacaaacctggtgaaaaataaatgcgttaat
tttaattttaacggtttgaccggcaccggggtccttactgagtca
aataaaaagttcctgccgtttcagcagtttggccgcgatattgcc
gatactacagacgcggtgcgggaccctcaaactctcgaaatcctg
gatataacgccttgctcttttggcggggtaagcgtcattacccca
ggtacaaatacgtcaaatcaggtcgctgttctgtaccaagacgtc
aattgcaccgaggtcccagtagctatccatgccgatcaactcacg
ccaacctggcgggtatactctacgggatccaacgtcttccaaacc
agggggggtgcctcatcggagccgaacacgtaaacaatagctatg
agtgcgatattcccataggggccggaatctgcgcttcataccaaa
cccagactaattctccccgacgagcccgaagcgtggcatctcaaa
gcataatagcgtacacgatgagcttgggagcagaaaactcagtgg
cgtatagcaataactccatcgcgataccaaccaattttacaatca
gtgtgactacagagatcttgccggttagtatgacaaagacttcag
tggattgcacgatgtacatctgtggagatagtaccgagtgcagca
accttttgttgcagtatggctcattttgtacccaactcaatcggg
ccctgaccggtattgcagttgaacaggataaaaacacacaagagg
tctttgcacaagttaaacaaatatacaagaccccgcctatcaaag
actttggtggttttaattttagccaaattcttcccgacccgtcaa
agccgagtaagcggtcattcatagaggatctcctctttaataagg
taaccttggctgacgcgggatttattaaacagtacggtgactgtt
tgggagatattgcagctcgagacttgatctgcgcccagaaattca
acggtctgactgttttgcctccgctcttgacggacgagatgatcg
cccagtatacttccgccttgctcgctggtacgatcacctccggct
ggacctttggcgctggagctgctctccagatcccattcgcgatgc
agatggcttaccgatttaacggtattggcgttacacaaaatgtat
tgtacgagaaccaaaaactcatcgctaatcaattcaactcagcta
ttggcaaaattcaagactcattgtcctctacagccagcgccttgg
gtaaattgcaagacgtagtgaaccaaaatgcacaggccttgaaca
ctctggtcaagcaactcagttccaattttggtgcaatctcatccg
ttctcaacgatatactgtctcgcctggataaggtggaagctgagg
tgcaaatcgacagactcataacgggaaggctgcagagtctgcaga
cctatgtcactcagcagcttattcgagcggccgaaatccgcgctt
ccgccaatttggctgctactaaaatgagcgaatgcgtactcgggc
aatctaaacgagtcgacttttgtggcaaaggctaccacctcatgt
catttccccagagtgcgccacacggggtggttttccttcatgtca
cctacgtgcccgcccaagaaaagaactttactaccgcaccggcca
tctgccatgatggaaaggcccatttcccgagagagggcgtatttg
tctcaaacggtactcactggtttgtgacacaacgcaatttctacg
aaccccaaattataaccactgacaacacatttgtctctggaaact
gtgatgtcgttattggtatagttaacaacacggtctacgacccac
tccagccagagctggactcttttaaagaagaactcgacaagtatt
ttaaaaatcacacgagtcccgacgttgaccttggagacattagtg
ggatcaacgcgagtgtcgttaacatccaaaaggagattgatcgcc
tcaatgaggtggccaagaatctcaacgaatccctcatagatctcc
aagagctcggtaagtatgagcagtatattaagtggccctggtata
tatggctcggctttatcgccggcctgattgcaatagttatggtta
cgataatgctgtgctgtatgacttcatgctgttcttgtttgaaag
gctgttgctcatgtgggagttgttgcaagtttgatgaggacgaca
gtgaacccgtattgaagggtgtgaagcttcattacacttaa
Signal Domain
(SEQ ID NO: 3)
MFVFLVLLPLVSS
N-terminus Domain
(SEQ ID NO: 4)
QCVNLTTRTQLPPAYTNSFTRGVYYPDKVFRSSVLHSTQDLFLPF
FSNVTWFHAIHVSGTNGTKRFDNPVLPFNDGVYFASTEKSNIIRG
WIFGTTLDSKTQSLLIVNNATNVVIKVCEFQFCNDPFLGVYYHKN
NKSWMESEFRVYSSANNCTFEYVSQPFLMDLEGKQGNFKNLREFV
FKNIDGYFKIYSKHTPINLVRDLPQGFSALEPLVDLPIGINITRF
QTLLALHRSYLTPGDSSSGWTAGAAAYYVGYLQPRTFLLKYNENG
TITDAVDCALDPLSETKCTLKSFTVEKGIYQTSNF
Receptor Binding Domain
(SEQ ID NO: 5)
RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYLY
RLFRKSNLKPFERDISTEIYQAGSTPCNGVEGENCYFPLQSYGFQ
PTNGVGYQPYRV
S2 Domain
(SEQ ID NO: 6)
VVLSFELLHAPATVCGPKKSTNLVKNKCVNFNFNGLTGTGVLTES
NKKFLPFQQFGRDIADTTDAVRDPQTLEILDITPCSFGGVSVITP
GTNTSNQVAVLYQDVNCTEVPVAIHADQLTPTWRVYSTGSNVFQT
RAGCLIGAEHVNNSYECDIPIGAGICASYQTQTNSPRRARSVASQ
SIIAYTMSLGAENSVAYSNNSIAIPTNFTISVTTEILPVSMTKTS
VDCTMYICGDSTECSNLLLQYGSFCTQLNRALTGIAVEQDKNTQE
VFAQVKQIYKTPPIKDFGGFNFSQILPDPSKPSKRSFIEDLLFNK
VTLADAGFIKQYGDCLGDIAARDLICAQKFNGLTVLPPLLTDEMI
AQYTSALLAGTITSGWTFGAGAALQIPFAMQMAYRENGIGVTQNV
LYENQKLIANQFNSAIGKIQDSLSSTASALGKLQDVVNQNAQALN
TLVKQLSSNFGAISSVLNDILSRLDKVEAEVQIDRLITGRLQSLQ
TYVTQQLIRAAEIRASANLAATKMSECVLGQSKRVDFCGKGYHLM
SFPQSAPHGVVFLHVTYVPAQEKNFTTAPAICHDGKAHFPREGVF
VSNGTHWFVTQRNFYEPQIITTDNTFVSGNCDVVIGIVNNTVYDP
LQPELDSFKEELDKYFKNHTSPDVDLGDISGINASVVNIQKEIDR
LNEVAKNLNESLIDLQELGKYEQ
Transmembrane Region
(SEQ ID NO: 7)
YIKWPWY
Cytoplasmic Tail
(SEQ ID NO: 8)
IWLGFIAGLIAIVMVTIMLCCMTSCCSCLKGCCSCGSCCKFDEDD
SEPVLKGVKLHYT
CB-107-COVID-19 Spike Glycoprotein
(S-protein) (Nucleotide Sequence)
SEQ ID NO: 9
atgtttgtgttcctggtgcttctgccacttgtttcctcacaatgc
gtcaatctcacaacaagaacgcagcttccgcctgcatatacaaat
agtttcaccagaggcgtttattatccagataaggtctttcgaagc
tctgtcctccattctacgcaagatctgttcttgccattcttttca
aacgtgacgtggtttcacgctattcacgtttctggcactaatggc
acgaagcgctttgacaaccccgtccttccgttcaacgatggcgtc
tatttcgcgagtacagagaagtcaaatataatccgcggctggata
tttggcacaaccctcgatagtaaaacacaatcccttctgatcgta
aataacgcaacgaatgtggtgattaaggtatgcgagtttcaattt
tgcaacgacccgttccttggcgtttattatcacaaaaataacaag
tcctggatggagtccgaattcagagtgtattcttctgccaacaat
tgcacctttgagtacgtgtcccagcccttcttgatggatctggaa
ggaaaacagggcaatttcaagaatttgagagagttcgtcttcaaa
aacatcgatggttattttaagatctatagcaagcacaccccgata
aacctcgtacgggaccttccccaggggtttagcgcactggagcct
cttgtagatctcccgattggcatcaatataaccaggtttcaaact
cttcttgcacttcaccgaagctatcttacgcccggcgatagctct
tccggttggacagccggcgccgcggcctactatgtgggatatctc
cagcctagaacctttctgcttaagtacaatgaaaacggcactatt
acagacgccgtagattgcgctctggatcctcttagcgaaactaag
tgtactctgaaatcttttacggtagagaagggaatttatcaaaca
agtaatttccgggtacaacccacagagagcatcgtccggtttccg
aacattacgaatctttgcccgtttggcgaagtcttcaatgcaaca
cgcttcgcttccgtttatgcgtggaatcgcaagaggatctctaac
tgcgttgctgactacagtgttctttataacagcgctagcttttct
acattcaaatgttacggggtaagccccaccaagctcaacgacttg
tgttttaccaatgtttatgcggattctttcgtgattcgcggtgat
gaagttaggcagattgcgccaggacagactgggaagattgccgac
tataactataagctccctgacgacttcactgggtgtgtcattgct
tggaacagcaacaatctcgacagcaaagttggaggcaactataat
tatctctaccggctttttcgcaagtctaacctcaagcccttcgaa
agagacatcagtactgagatctatcaagcaggaagcacgccatgc
aacggtgtcgagggcttcaactgttattttcctcttcaatcctac
ggctttcaaccgacaaacggcgtcggatatcagccttatcgagtc
gttgtactttctttcgaacttctgcacgcaccagctactgtctgc
gggcctaaaaaatctacaaacctggtgaaaaataaatgcgttaat
tttaattttaacggtttgaccggcaccggggtccttactgagtca
aataaaaagttcctgccgtttcagcagtttggccgcgatattgcc
gatactacagacgcggtgcgggaccctcaaactctcgaaatcctg
gatataacgccttgctcttttggggggtaagcgtcattaccccag
gtacaaatacgtcaaatcaggtcgctgttctgtaccaagacgtca
attgcaccgaggtcccagtagctatccatgccgatcaactcacgc
caacctggcgggtatactctacgggatccaacgtcttccaaacca
gggggggtgcctcatcggagccgaacacgtaaacaatagctatga
gtgcgatattcccataggggccggaatctgcgcttcataccaaac
ccagactaattctccccgacgagcccgaagcgtggcatctcaaag
cataatagcgtacacgatgagcttgggagcagaaaactcagtggc
gtatagcaataactccatcgcgataccaaccaattttacaatcag
tgtgactacagagatcttgccggttagtatgacaaagacttcagt
ggattgcacgatgtacatctgtggagatagtaccgagtgcagcaa
ccttttgttgcagtatggctcattttgtacccaactcaatcgggc
cctgaccggtattgcagttgaacaggataaaaacacacaagaggt
ctttgcacaagttaaacaaatatacaagaccccgcctatcaaaga
ctttggtggttttaattttagccaaattcttcccgacccgtcaaa
gccgagtaagcggtcattcatagaggatctcctctttaataaggt
aaccttggctgacgcgggatttattaaacagtacggtgactgttt
gggagatattgcagctcgagacttgatctgcgcccagaaattcaa
cggtctgactgttttgcctccgctcttgacggacgagatgatcgc
ccagtatacttccgccttgctcgctggtacgatcacctccggctg
gacctttggcgctggagctgctctccagatcccattcgcgatgca
gatggcttaccgatttaacggtattggcgttacacaaaatgtatt
gtacgagaaccaaaaactcatcgctaatcaattcaactcagctat
tggcaaaattcaagactcattgtcctctacagccagcgccttggg
taaattgcaagacgtagtgaaccaaaatgcacaggccttgaacac
tctggtcaagcaactcagttccaattttggtgcaatctcatccgt
tctcaacgatatactgtctcgcctggataaggtggaagctgaggt
gcaaatcgacagactcataacgggaaggctgcagagtctgcagac
ctatgtcactcagcagcttattcgagcggccgaaatccgcgcttc
cgccaatttggctgctactaaaatgagcgaatgcgtactcgggca
atctaaacgagtcgacttttgtggcaaaggctaccacctcatgtc
atttccccagagtgcgccacacggggtggttttccttcatgtcac
ctacgtgcccgcccaagaaaagaactttactaccgcaccggccat
ctgccatgatggaaaggcccatttcccgagagagggcgtatttgt
ctcaaacggtactcactggtttgtgacacaacgcaatttctacga
accccaaattataaccactgacaacacatttgtctctggaaactg
tgatgtcgttattggtatagttaacaacacggtctacgacccact
ccagccagagctggactcttttaaagaagaactcgacaagtattt
taaaaatcacacgagtcccgacgttgaccttggagacattagtgg
gatcaacgcgagtgtgttaacatccaaaaggagattgatcgcctc
aatgaggtggccaagaatctcaacgaatccctcatagatctccaa
gagctcggtaagtatgagcagtataa
Signal Domain
(SEQ ID NO: 10)
MFVFLVLLPLVSS
N-terminus Domain
(SEQ ID NO: 11)
QCVNLTTRTQLPPAYTNSFTRGVYYPDKVFRSSVLHSTQDLFLPF
FSNVTWFHAIHVSGTNGTKRFDNPVLPFNDGVYFASTEKSNIIRG
WIFGTTLDSKTQSLLIVNNATNVVIKVCEFQFCNDPFLGVYYHKN
NKSWMESEFRVYSSANNCTFEYVSQPFLMDLEGKQGNFKNLREFV
FKNIDGYFKIYSKHTPINLVRDLPQGFSALEPLVDLPIGINITRF
QTLLALHRSYLTPGDSSSGWTAGAAAYYVGYLQPRTFLLKYNENG
TITDAVDCALDPLSETKCTLKSFTVEKGIYQTSNF
Receptor Binding Domain
(SEQ ID NO: 12)
RVQPTESIVRFPNITNLCPFGEVENATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYLY
RLFRKSNLKPFERDISTEIYQAGSTPCNGVEGENCYFPLQSYGFQ
PTNGVGYQPYRV
S2 Domain
(SEQ ID NO: 13)
VVLSFELLHAPATVCGPKKSTNLVKNKCVNFNFNGLTGTGVLTES
NKKFLPFQQFGRDIADTTDAVRDPQTLEILDITPCSFGGVSVITP
GTNTSNQVAVLYQDVNCTEVPVAIHADQLTPTWRVYSTGSNVFQT
RAGCLIGAEHVNNSYECDIPIGAGICASYQTQTNSPRRARSVASQ
SIIAYTMSLGAENSVAYSNNSIAIPTNFTISVTTEILPVSMTKTS
VDCTMYICGDSTECSNLLLQYGSFCTQLNRALTGIAVEQDKNTQE
VFAQVKQIYKTPPIKDFGGFNFSQILPDPSKPSKRSFIEDLLFNK
VTLADAGFIKQYGDCLGDIAARDLICAQKENGLTVLPPLLTDEMI
AQYTSALLAGTITSGWTFGAGAALQIPFAMQMAYRENGIGVTQNV
LYENQKLIANQFNSAIGKIQDSLSSTASALGKLQDVVNQNAQALN
TLVKQLSSNFGAISSVLNDILSRLDKVEAEVQIDRLITGRLQSLQ
TYVTQQLIRAAEIRASANLAATKMSECVLGQSKRVDFCGKGYHLM
SFPQSAPHGVVFLHVTYVPAQEKNFTTAPAICHDGKAHFPREGVF
VSNGTHWFVTQRNFYEPQIITTDNTFVSGNCDVVIGIVNNTVYDP
LQPELDSFKEELDKYFKNHTSPDVDLGDISGINASVVNIQKEIDR
LNEVAKNLNESLIDLQELGKYEQ
South African Variant RBD domain
(SEQ ID NO: 14)
atgcgggtacaacccacagagagcatcgtccggtttccgaacatt
acgaatctttgcccgtttggcgaagtcttcaatgcaacacgcttc
gcttccgtttatgcgtggaatcgcaagaggatctctaactgcgtt
gctgactacagtgttctttataacagcgctagcttttctacattc
aaatgttacggggtaagccccaccaagctcaacgacttgtgtttt
accaatgtttatgcggattctttcgtgattcgcggtgatgaagtt
aggcagattgcgccaggacagactgggaatattgccgactataac
tataagctccctgacgacttcactgggtgtgtcattgcttggaac
agcaacaatctcgacagcaaagttggaggcaactataattatctc
taccggctttttcgcaagtctaacctcaagcccttcgaaagagac
atcagtactgagatctatcaagcaggaagcacgccatgcaacggt
gtcaagggcttcaactgttattttcctcttcaatcctacggcttt
caaccgacatacggcgtcggatatcagccttatcgagtctaa
Psi Element
(SEQ ID NO: 15)
gtggcgatcctgccctcatccgtctcgcttattcggggagcggac
gatgaccctagtagagggggctgcggcttaggagggcagaagctg
agtggcgtcggagggagctctactgcagggagcccagatacccta
ccgagaactcagagagtcgttggaagacgggaagg
Subgenomic Promoter
(SEQ ID NO: 16)
cctgaatggactacgacatagtctagtccgccaag
Amino acid sequence for CB-107- COVID-19
Spike Glycoprotein (S-protein) Ectodomain
SEQ ID NO: 17
MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNERVOPTESIVRFPNITNLCPFGEVENATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGENCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFT
ISVTTEILPVSMTKTSVDCTMYICGDSTECSNLLLQYGSFCTQLN
RALTGIAVEQDKNTQEVFAQVKQIYKTPPIKDFGGFNFSQILPDP
SKPSKRSFIEDLLFNKVTLADAGFIKQYGDCLGDIAARDLICAQK
FNGLTVLPPLLTDEMIAQYTSALLAGTITSGWTFGAGAALQIPFA
MQMAYRENGIGVTQNVLYENQKLIANQFNSAIGKIQDSLSSTASA
LGKLQDVVNQNAQALNTLVKQLSSNFGAISSVLNDILSRLDKVEA
EVQIDRLITGRLQSLQTYVTQQLIRAAEIRASANLAATKMSECVL
GQSKRVDFCGKGYHLMSFPQSAPHGVVFLHVTYVPAQEKNFTTAP
AICHDGKAHFPREGVFVSNGTHWFVTQRNFYEPQIITTDNTFVSG
NCDVVIGIVNNTVYDPLQPELDSFKEELDKYFKNHTSPDVDLGDI
SGINASVVNIQKEIDRLNEVAKNLNESLIDLQELGKYEQ

### 6. Equivalents and Incorporation by Reference

All references cited herein are incorporated by reference to the same extent as if each individual publication, database entry (e.g. Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

What is claimed is:

1. A storage stable composition of virus like particles (VLPs), the VLPs comprising:
a. an alphavirus replicon comprising a recombinant polynucleotide, wherein the polynucleotide encodes at least one polypeptide or fragment thereof which is an immunogenic antigen of an infectious agent,
b. a retroviral gag protein,
c. a fusogenic envelope protein,
d. a retroviral packaging signal,
e. wherein the alphavirus replicon further encodes nonstructural proteins NSP1, NSP2, NSP3, and NSP4 and one or more subgenomic promoters, and
wherein the VLP does not contain an alphavirus structural protein;
wherein the composition is stable for at least about one-six months at 4° C.-10° C.

2. The VLP of claim 1, wherein the infectious agent is a virus.

3. The VLP of claim 1 or 2, wherein the virus is a coronavirus.

4. The VLP of claim 3, wherein the coronavirus is SARS-CoV-2.

5. The VLP of any of claims 1-4, wherein the immunogenic antigen is a surface antigen.

6. The VLP of claim 5, wherein the surface antigen has at least 80% identity to a coronavirus spike protein, coronavirus membrane protein, coronavirus hemagglutinin esterase (HE), coronavirus envelope protein, or any combination thereof.

7. The VLP of claim 5, wherein the surface antigen has 80% identity to a coronavirus spike protein.

8. The VLP of claim 7, wherein the spike protein sequence has at least 90% identity with a coronavirus spike protein (SEQ ID NO:1) or ectodomain SEQ ID NO: 17, or any immunogenic fragment or combination thereof.

9. The VLP of claim 8, wherein the spike protein sequence comprises SEQ ID NO: 1 or the ectodomain region SEQ ID NO:17, or a combination thereof.

10. The VLP of claim 9, wherein the spike protein sequence is encoded by a codon optimized sequence of SEQ ID NO: 2; or a codon optimized sequence encoding the ectodomain sequence of SEQ ID NO: 17.

11. The VLP of claim 8, wherein the spike protein sequence comprises a sequence having at least 80% identity with a sequence selected from SEQ ID NOs: 3-8.

12. The VLP of claim 8, wherein the spike protein sequence comprises a sequence having at least 90% identity with a sequence selected from SEQ ID NO: 3-8.

13. The VLP of claim 8, wherein the spike protein sequences comprise two or more sequences each having at least 80% sequence identity with a sequence selected from SEQ ID NOs: 3-8.

14. The VLP of claim 8, wherein the spike protein sequence comprises two or more sequences selected from SEQ ID NOs: 3-8.

15. The VLP of claim 8, wherein the spike protein sequence comprises a signal domain, an N-terminus domain, a receptor binding domain (RBD), an S2 domain, a transmembrane region, a cytoplasmic tail, or a combination thereof.

16. The VLP of claim 8, wherein the spike protein sequence comprises a sequence having at least 80% identity with a sequence selected from SEQ ID NOs: 10-13.

17. The VLP of claim 8, wherein the spike protein sequence comprises a sequence having at least 90% identity with a sequence selected from SEQ ID NO: 10-13.

18. The VLP of claim 8, wherein the spike protein sequences comprise two or more sequences each having at least 80% sequence identity with a sequence selected from SEQ ID NOs: 10-13.

19. The VLP of claim 8, wherein the spike protein sequence comprises two or more sequences selected from SEQ ID NOs: 10-13.

20. The VLP of any of the previous claims, wherein at least one psi (P) element is present upstream of the alphavirus replicon, and the psi element is from any member of the Retroviridae family.

21. The VLP of claim 20, wherein the at least one psi (P) element is derived from Rous sarcoma virus (RSV).

22. The VLP of any of the previous claims, wherein the virus nonstructural proteins NSP1, NSP2, NSP3, and NSP4 are from Sindbis virus or Venezuelan equine encephalitis virus; and, optionally, the retroviral packaging signal is derived from Rous sarcoma virus (RSV).

23. The VLP of any of the previous claims, wherein the alphavirus is a Venezuelan equine encephalitis virus.

24. The VLP of any of the previous claims, wherein the VLP does not comprise or express a retroviral pol gene.

25. The VLP of any of the previous claims, wherein the retroviral gag protein is derived from Rous sarcoma virus (RSV).

26. The VLP of any of the previous claims, wherein the fusogenic envelope protein is a glycoprotein, or fragment or derivative thereof.

27. The VLP of any of the previous claims, wherein the fusogenic envelope protein is VSV-G and is capable of binding to a target immune cell or antigen presenting cell.

28. The VLP of claim 27, wherein the target immune cell is a dendritic cell.

29. The VLP of any of the previous claims, wherein the VLP is capable of binding to a target cell of a subject, after which the target cell is capable of expressing the immunogenic antigen of the infectious agent, which expression is capable of producing an immune response in the subject, including a T cell response against coronavirus in the subject.

30. The VLP of claim 29, wherein the immune response including a T cell response is induced by a dosing regimen comprising one or two administrations of the VLP, wherein the dose is sufficient to induce an immune response against coronavirus in a subject.

31. The VLP of claim 30, wherein the dosing regimen comprises one administration of the VLP, wherein the one dose administration is sufficient to induce an immune response against the coronavirus in a subject.

32. A pharmaceutical composition comprising the VLP of any of the previous claims.

33. The pharmaceutical composition of claim 32, wherein the composition further comprises a pharmaceutically acceptable carrier, diluent, additive, or any combination thereof.

34. The pharmaceutical composition of claim 32 or 33, which is capable of inducing an immune response against coronavirus in a mammalian subject.

35. The pharmaceutical composition of any one of claims 32-34, wherein following administration of the composition to the subject, the VLP is capable of inducing a T cell response against coronavirus in a mammalian subject.

36. The pharmaceutical composition of claim 35, wherein the coronavirus is SARS-CoV-2.

37. The pharmaceutical composition of claim 36 in a dosage formulation wherein the dose of the VLP is equivalent to about 100 pg to 1000 ng of RNA.

38. The VLP or pharmaceutical composition of any of claims 1-37, for use in diminishing or preventing at least one symptom of a coronavirus infection in a mammalian subject.

39. The VLP or pharmaceutical composition of claim 38, wherein said diminishing or preventing comprises inducing coronavirus-specific immunity against SARS-CoV-2 (COVID-19).

40. The VLP or pharmaceutical composition of any of claims 1-39 for use in inducing cellular and or humoral immunity in a mammalian subject.

41. The VLP or pharmaceutical composition of any of claims 1-40 for use in eliciting an immune response in a mammalian subject.

42. The VLP or pharmaceutical composition of claim 41, wherein said inducing or eliciting an immune response is an immune response against SARS-CoV-2 (COVID-19).

43. The VLP or pharmaceutical composition of any of claims 1-42 for use in inducing neutralizing antibodies against SARS-CoV-2 in a mammalian subject.

44. A method of diminishing or preventing a coronavirus infection in a mammalian subject comprising administering the VLP of any of claims 1-31, or the pharmaceutical composition of any of claims 32-43 to the subject.

45. A method of inducing cellular and or humoral immunity against a coronavirus in a mammalian subject, comprising administering the VLP of any of claims 1-31 or the pharmaceutical composition of any of claims 32-43 to the mammalian subject.

46. A method of eliciting an immune response against a coronavirus in a mammalian subject, comprising administering the VLP of any of claims 1-31, or the pharmaceutical composition of any of claims 32-43 to the subject.

47. A method of inducing neutralizing antibodies against SARS-CoV-2 in a mammalian subject, comprising administering the VLP of any of claims 1-32, or the pharmaceutical composition of any of claims 33-43 to the subject.

48. The method of any one of claims 44-47, wherein the method further includes inducing a T cell response against the coronavirus.

49. The method of any one of claims 44-48, wherein the immune response is induced by a regimen comprising one or two administrations of the VLP or pharmaceutical composition.

50. The method of any one of claims 44-49, wherein the immune response is induced by a regimen comprising one administration of the VLP or pharmaceutical composition.

51. The method of any one of claims 44-50, wherein the coronavirus is SARS-CoV-2.

52. A method of expressing a recombinant polynucleotide encoding an immunogenic antigen of an infectious agent in a subject, comprising administering the VLP of any of claims 1-31, or the pharmaceutical composition of any of claims 32-43 to the subject.

53. The method of claim 54, wherein the VLP is capable of binding to a target cell, after which the target cell is capable of expressing the immunogenic antigen of the infectious agent, which expression is capable of inducing a cellular and/or humoral immune response to SARS-CoV-2 in the subject.

54. A method of producing the VLP of any of claims 1-31, comprising:
a. co-transforming a eukaryotic cell with:
a first plasmid comprising a polynucleotide sequence encoding the alphavirus replicon, wherein the polynucleotide encodes at least one polypeptide or fragment thereof which is an immunogenic antigen of an infectious agent;
a second plasmid comprising a polynucleotide sequence encoding a retroviral gag protein; and
a third plasmid comprising a polynucleotide sequence encoding the fusogenic envelope protein;
b. culturing the co-transformed eukaryotic cell under conditions suitable to cause each plasmid to produce its encoded product, thereby producing the VLP; and
c. isolating the VLP from the eukaryotic cell.

55. A VLP produced by the method of claim 54.

56. A method of diminishing or preventing a coronavirus infection in a mammalian subject comprising administering the VLP of claim 55 to the subject.

57. A method of inducing cellular and or humoral immunity in a mammalian subject, comprising administering the VLP of claim 55 to the subject.

58. A method of eliciting an immune response in a subject, comprising administering the VLP of claim 55 to the subject.

59. A method of inducing neutralizing antibodies against SARS-CoV-2 in a subject, comprising administering the VLP of claim 55 to the subject.

60. The method of any one of claims 56-59, wherein the method further includes inducing a T cell response against the coronavirus.

61. The method of claim 60, wherein the T cell response is induced by a regimen comprising one or at least two administrations.

62. The method of claim 61, wherein the coronavirus is SARS-CoV-2 (COVID-19).

63. A method of expressing a heterologous nucleic acid sequence in the host, comprising administering to a subject the VLP of claim 55, wherein the VLP is capable of binding to a host cell, after which the host cell is capable of expressing the immunogenic antigen of the infectious agent, which expression is capable of inducing a cellular and/or humoral immune response to SARS-CoV-2 in the subject.

64. The VLP or composition of claim 55, wherein the VLP is stable for at least about six to nine months.

65. The VLP or composition of claim 64, wherein the VLP is stable for at least about nine to twelve months.

66. The VLP or composition of any of claims 1-43, wherein the VLP is stable at about −80° C. for at least about one year.

67. The VLP or composition of claim 66, wherein the VLP is stable for about two years.

68. The VLP or composition of claim 67, wherein the VLP is stable for about three years.

69. The method of any of claims 44-53 or 56-63, wherein the dose of the VLP is equivalent to about 100 pg to 1000 ng of RNA.

70. The VLP or composition of any of claims 1-43, wherein the VLP is capable of binding to a host cell, after which the host cell is capable of expressing the immunogenic antigen of the infectious agent, which expression is capable of inducing a cellular and/or humoral immune response to Severe Acute Respiratory Syndrome (SARS-CoV), SARS-CoV-2, and variants of SARS-CoV-2 in the subject.


SEQUENCE LISTING (17 sequences)

SEQ ID NO: 1 - protein, 1273 aa, Artificial sequence
  synthetic sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3822 bp, Artificial sequence
  synthetic sequence
atgtttgtgttcctggtgcttctgccacttgtttcctcacaatgcgtcaatctcacaacaagaacgcagcttccgcctgcatatacaaatagtttcaccagaggcgtttattatccagataaggtctttcgaagctctgtcctccattctacgcaagatctgttcttgccattcttttcaaacgtgacgtggtttcacgctattcacgtttctggcactaatggcacgaagcgctttgacaaccccgtccttccgttcaacgatggcgtctatttcgcgagtacagagaagtcaaatataatccgcggctggatatttggcacaaccctcgatagtaaaacacaatcccttctgatcgtaaataacgcaacgaatgtggtgattaaggtatgcgagtttcaattttgcaacgacccgttccttggcgtttattatcacaaaaataacaagtcctggatggagtccgaattcagagtgtattcttctgccaacaattgcacctttgagtacgtgtcccagcccttcttgatggatctggaaggaaaacagggcaatttcaagaatttgagagagttcgtcttcaaaaacatcgatggttattttaagatctatagcaagcacaccccgataaacctcgtacgggaccttccccaggggtttagcgcactggagcctcttgtagatctcccgattggcatcaatataaccaggtttcaaactcttcttgcacttcaccgaagctatcttacgcccggcgatagctcttccggttggacagccggcgccgcggcctactatgtgggatatctccagcctagaacctttctgcttaagtacaatgaaaacggcactattacagacgccgtagattgcgctctggatcctcttagcgaaactaagtgtactctgaaatcttttacggtagagaagggaatttatcaaacaagtaatttccgggtacaacccacagagagcatcgtccggtttccgaacattacgaatctttgcccgtttggcgaagtcttcaatgcaacacgcttcgcttccgtttatgcgtggaatcgcaagaggatctctaactgcgttgctgactacagtgttctttataacagcgctagcttttctacattcaaatgttacggggtaagccccaccaagctcaacgacttgtgttttaccaatgtttatgcggattctttcgtgattcgcggtgatgaagttaggcagattgcgccaggacagactgggaagattgccgactataactataagctccctgacgacttcactgggtgtgtcattgcttggaacagcaacaatctcgacagcaaagttggaggcaactataattatctctaccggctttttcgcaagtctaacctcaagcccttcgaaagagacatcagtactgagatctatcaagcaggaagcacgccatgcaacggtgtcgagggcttcaactgttattttcctcttcaatcctacggctttcaaccgacaaacggcgtcggatatcagccttatcgagtcgttgtactttctttcgaacttctgcacgcaccagctactgtctgcgggcctaaaaaatctacaaacctggtgaaaaataaatgcgttaattttaattttaacggtttgaccggcaccggggtccttactgagtcaaataaaaagttcctgccgtttcagcagtttggccgcgatattgccgatactacagacgcggtgcgggaccctcaaactctcgaaatcctggatataacgccttgctcttttggcggggtaagcgtcattaccccaggtacaaatacgtcaaatcaggtcgctgttctgtaccaagacgtcaattgcaccgaggtcccagtagctatccatgccgatcaactcacgccaacctggcgggtatactctacgggatccaacgtcttccaaaccagggcggggtgcctcatcggagccgaacacgtaaacaatagctatgagtgcgatattcccataggggccggaatctgcgcttcataccaaacccagactaattctccccgacgagcccgaagcgtggcatctcaaagcataatagcgtacacgatgagcttgggagcagaaaactcagtggcgtatagcaataactccatcgcgataccaaccaattttacaatcagtgtgactacagagatcttgccggttagtatgacaaagacttcagtggattgcacgatgtacatctgtggagatagtaccgagtgcagcaaccttttgttgcagtatggctcattttgtacccaactcaatcgggccctgaccggtattgcagttgaacaggataaaaacacacaagaggtctttgcacaagttaaacaaatatacaagaccccgcctatcaaagactttggtggttttaattttagccaaattcttcccgacccgtcaaagccgagtaagcggtcattcatagaggatctcctctttaataaggtaaccttggctgacgcgggatttattaaacagtacggtgactgtttgggagatattgcagctcgagacttgatctgcgcccagaaattcaacggtctgactgttttgcctccgctcttgacggacgagatgatcgcccagtatacttccgccttgctcgctggtacgatcacctccggctggacctttggcgctggagctgctctccagatcccattcgcgatgcagatggcttaccgatttaacggtattggcgttacacaaaatgtattgtacgagaaccaaaaactcatcgctaatcaattcaactcagctattggcaaaattcaagactcattgtcctctacagccagcgccttgggtaaattgcaagacgtagtgaaccaaaatgcacaggccttgaacactctggtcaagcaactcagttccaattttggtgcaatctcatccgttctcaacgatatactgtctcgcctggataaggtggaagctgaggtgcaaatcgacagactcataacgggaaggctgcagagtctgcagacctatgtcactcagcagcttattcgagcggccgaaatccgcgcttccgccaatttggctgctactaaaatgagcgaatgcgtactcgggcaatctaaacgagtcgacttttgtggcaaaggctaccacctcatgtcatttccccagagtgcgccacacggggtggttttccttcatgtcacctacgtgcccgcccaagaaaagaactttactaccgcaccggccatctgccatgatggaaaggcccatttcccgagagagggcgtatttgtctcaaacggtactcactggtttgtgacacaacgcaatttctacgaaccccaaattataaccactgacaacacatttgtctctggaaactgtgatgtcgttattggtatagttaacaacacggtctacgacccactccagccagagctggactcttttaaagaagaactcgacaagtattttaaaaatcacacgagtcccgacgttgaccttggagacattagtgggatcaacgcgagtgtcgttaacatccaaaaggagattgatcgcctcaatgaggtggccaagaatctcaacgaatccctcatagatctccaagagctcggtaagtatgagcagtatattaagtggccctggtatatatggctcggctttatcgccggcctgattgcaatagttatggttacgataatgctgtgctgtatgacttcatgctgttcttgtttgaaaggctgttgctcatgtgggagttgttgcaagtttgatgaggacgacagtgaacccgtattgaagggtgtgaagcttcattacacttaa

SEQ ID NO: 3 - protein, 13 aa, Artificial sequence
  synthetic sequence
MFVFLVLLPLVSS

SEQ ID NO: 4 - protein, 305 aa, Artificial sequence
  synthetic sequence
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNF

SEQ ID NO: 5 - protein, 192 aa, Artificial sequence
  synthetic sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 6 - protein, 698 aa, Artificial sequence
  synthetic sequence
VVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 7 - protein, 7 aa, Artificial sequence
  synthetic sequence
YIKWPWY

SEQ ID NO: 8 - protein, 58 aa, Artificial sequence
  synthetic sequence
IWLGFIAGLIAIVMVTIMLCCMTSCCSCLKGCCSCGSCCKFDEDDSEPVLKGVKLHYT

SEQ ID NO: 9 - DNA, 3629 bp, Artificial sequence
  synthetic sequence
atgtttgtgttcctggtgcttctgccacttgtttcctcacaatgcgtcaatctcacaacaagaacgcagcttccgcctgcatatacaaatagtttcaccagaggcgtttattatccagataaggtctttcgaagctctgtcctccattctacgcaagatctgttcttgccattcttttcaaacgtgacgtggtttcacgctattcacgtttctggcactaatggcacgaagcgctttgacaaccccgtccttccgttcaacgatggcgtctatttcgcgagtacagagaagtcaaatataatccgcggctggatatttggcacaaccctcgatagtaaaacacaatcccttctgatcgtaaataacgcaacgaatgtggtgattaaggtatgcgagtttcaattttgcaacgacccgttccttggcgtttattatcacaaaaataacaagtcctggatggagtccgaattcagagtgtattcttctgccaacaattgcacctttgagtacgtgtcccagcccttcttgatggatctggaaggaaaacagggcaatttcaagaatttgagagagttcgtcttcaaaaacatcgatggttattttaagatctatagcaagcacaccccgataaacctcgtacgggaccttccccaggggtttagcgcactggagcctcttgtagatctcccgattggcatcaatataaccaggtttcaaactcttcttgcacttcaccgaagctatcttacgcccggcgatagctcttccggttggacagccggcgccgcggcctactatgtgggatatctccagcctagaacctttctgcttaagtacaatgaaaacggcactattacagacgccgtagattgcgctctggatcctcttagcgaaactaagtgtactctgaaatcttttacggtagagaagggaatttatcaaacaagtaatttccgggtacaacccacagagagcatcgtccggtttccgaacattacgaatctttgcccgtttggcgaagtcttcaatgcaacacgcttcgcttccgtttatgcgtggaatcgcaagaggatctctaactgcgttgctgactacagtgttctttataacagcgctagcttttctacattcaaatgttacggggtaagccccaccaagctcaacgacttgtgttttaccaatgtttatgcggattctttcgtgattcgcggtgatgaagttaggcagattgcgccaggacagactgggaagattgccgactataactataagctccctgacgacttcactgggtgtgtcattgcttggaacagcaacaatctcgacagcaaagttggaggcaactataattatctctaccggctttttcgcaagtctaacctcaagcccttcgaaagagacatcagtactgagatctatcaagcaggaagcacgccatgcaacggtgtcgagggcttcaactgttattttcctcttcaatcctacggctttcaaccgacaaacggcgtcggatatcagccttatcgagtcgttgtactttctttcgaacttctgcacgcaccagctactgtctgcgggcctaaaaaatctacaaacctggtgaaaaataaatgcgttaattttaattttaacggtttgaccggcaccggggtccttactgagtcaaataaaaagttcctgccgtttcagcagtttggccgcgatattgccgatactacagacgcggtgcgggaccctcaaactctcgaaatcctggatataacgccttgctcttttggcggggtaagcgtcattaccccaggtacaaatacgtcaaatcaggtcgctgttctgtaccaagacgtcaattgcaccgaggtcccagtagctatccatgccgatcaactcacgccaacctggcgggtatactctacgggatccaacgtcttccaaaccagggcggggtgcctcatcggagccgaacacgtaaacaatagctatgagtgcgatattcccataggggccggaatctgcgcttcataccaaacccagactaattctccccgacgagcccgaagcgtggcatctcaaagcataatagcgtacacgatgagcttgggagcagaaaactcagtggcgtatagcaataactccatcgcgataccaaccaattttacaatcagtgtgactacagagatcttgccggttagtatgacaaagacttcagtggattgcacgatgtacatctgtggagatagtaccgagtgcagcaaccttttgttgcagtatggctcattttgtacccaactcaatcgggccctgaccggtattgcagttgaacaggataaaaacacacaagaggtctttgcacaagttaaacaaatatacaagaccccgcctatcaaagactttggtggttttaattttagccaaattcttcccgacccgtcaaagccgagtaagcggtcattcatagaggatctcctctttaataaggtaaccttggctgacgcgggatttattaaacagtacggtgactgtttgggagatattgcagctcgagacttgatctgcgcccagaaattcaacggtctgactgttttgcctccgctcttgacggacgagatgatcgcccagtatacttccgccttgctcgctggtacgatcacctccggctggacctttggcgctggagctgctctccagatcccattcgcgatgcagatggcttaccgatttaacggtattggcgttacacaaaatgtattgtacgagaaccaaaaactcatcgctaatcaattcaactcagctattggcaaaattcaagactcattgtcctctacagccagcgccttgggtaaattgcaagacgtagtgaaccaaaatgcacaggccttgaacactctggtcaagcaactcagttccaattttggtgcaatctcatccgttctcaacgatatactgtctcgcctggataaggtggaagctgaggtgcaaatcgacagactcataacgggaaggctgcagagtctgcagacctatgtcactcagcagcttattcgagcggccgaaatccgcgcttccgccaatttggctgctactaaaatgagcgaatgcgtactcgggcaatctaaacgagtcgacttttgtggcaaaggctaccacctcatgtcatttccccagagtgcgccacacggggtggttttccttcatgtcacctacgtgcccgcccaagaaaagaactttactaccgcaccggccatctgccatgatggaaaggcccatttcccgagagagggcgtatttgtctcaaacggtactcactggtttgtgacacaacgcaatttctacgaaccccaaattataaccactgacaacacatttgtctctggaaactgtgatgtcgttattggtatagttaacaacacggtctacgacccactccagccagagctggactcttttaaagaagaactcgacaagtattttaaaaatcacacgagtcccgacgttgaccttggagacattagtgggatcaacgcgagtgtcgttaacatccaaaaggagattgatcgcctcaatgaggtggccaagaatctcaacgaatccctcatagatctccaagagctcggtaagtatgagcagtataa

SEQ ID NO: 10 - protein, 13 aa, Artificial sequence
  synthetic sequence
MFVFLVLLPLVSS

SEQ ID NO: 11 - protein, 305 aa, Artificial sequence; identical to SEQ ID NO: 4
  synthetic sequence

SEQ ID NO: 12 - protein, 192 aa, Artificial sequence; identical to SEQ ID NO: 5
  synthetic sequence

SEQ ID NO: 13 - protein, 698 aa, Artificial sequence; identical to SEQ ID NO: 6
  synthetic sequence

SEQ ID NO: 14 - DNA, 582 bp, Artificial sequence
  synthetic sequence
atgcgggtacaacccacagagagcatcgtccggtttccgaacattacgaatctttgcccgtttggcgaagtcttcaatgcaacacgcttcgcttccgtttatgcgtggaatcgcaagaggatctctaactgcgttgctgactacagtgttctttataacagcgctagcttttctacattcaaatgttacggggtaagccccaccaagctcaacgacttgtgttttaccaatgtttatgcggattctttcgtgattcgcggtgatgaagttaggcagattgcgccaggacagactgggaatattgccgactataactataagctccctgacgacttcactgggtgtgtcattgcttggaacagcaacaatctcgacagcaaagttggaggcaactataattatctctaccggctttttcgcaagtctaacctcaagcccttcgaaagagacatcagtactgagatctatcaagcaggaagcacgccatgcaacggtgtcaagggcttcaactgttattttcctcttcaatcctacggctttcaaccgacatacggcgtcggatatcagccttatcgagtctaa

SEQ ID NO: 15 - DNA, 170 bp, Artificial sequence
  synthetic sequence
gtggcgatcctgccctcatccgtctcgcttattcggggagcggacgatgaccctagtagagggggctgcggcttaggagggcagaagctgagtggcgtcggagggagctctactgcagggagcccagataccctaccgagaactcagagagtcgttggaagacgggaagg

SEQ ID NO: 16 - DNA, 35 bp, Artificial sequence
  synthetic sequence
cctgaatggactacgacatagtctagtccgccaag

SEQ ID NO: 17 - protein, 1208 aa, Artificial sequence
  synthetic sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ